(19) United States
(12) Patent Application Publication
(10) Pub. No.: US 2011/0009440 A1
(43) Pub. Date: Jan. 13, 2011

(54) GLYT1 TRANSPORTER INHIBITORS AND USES THEREOF IN TREATMENT OF NEUROLOGICAL AND NEUROPSYCHIATRIC DISORDERS

(76) Inventor: Howard Robert Marshall, Essex (GB)

Correspondence Address: GlaxoSmithKline;GLOBAL PATENTS -US, UW2220, P. O. BOX 1539, KING OF PRUSSIA, PA, 19406-0939, US

(21) Appl. No.: 12/524,586

(22) PCT Filed: Jan. 30, 2008
(86) PCT No.: PCT/EP08/51086

§ 371 Date: Jul. 27, 2009

(30) Foreign Application Priority Data
Feb. 1, 2007 (GB) 0701955.7

(51) Int. Cl. A61K 31/4439; C07D 235/02; C07D 409/12; C07D 401/06; C07D 401/14; C07D 405/14; C07D 403/12; C07D 401/12; C07D 409/14; C07D 417/14; A61K 31/4184; A61K 31/444; A61K 31/427; A61P 25/18; A61P 25/28; A61P 25/00

(52) U.S. Cl. 514/278; 548/300.7; 546/15; 548/147; 514/387; 514/365

(57) ABSTRACT

Compounds of formula (I) or a salt thereof are provided:

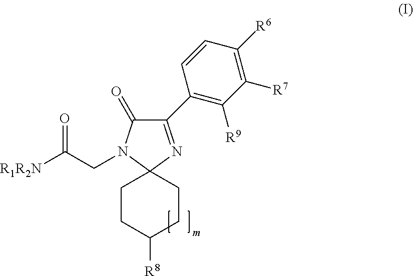

wherein R¹, R², R⁶, R⁷, R⁸, R⁹ and m are as defined in the description. Uses of the compounds as medicaments, and in the manufacture of medicament for treating neurological and neuropsychiatric disorders, in particular psychoses, dementia or attention deficit disorder are also disclosed. The invention further comprises pharmaceutical formulations comprising these compounds and combinations of these compounds and one or more other therapeutic agents.

The present invention relates to compounds, processes for their preparation, pharmaceutical compositions and medicaments containing them and to their use in treating disorders mediated by GlyT1, including neurological and neuropsychiatric disorders, in particular psychoses, dementia or attention deficit disorder.

Molecular cloning has revealed the existence in mammalian brains of two classes of glycine transporters, termed GlyT1 and GlyT2. GlyT1 is found predominantly in the forebrain and its distribution corresponds to that of glutaminergic pathways and NMDA receptors (Smith, et al., Neuron, 8, 1992: 927-935). Molecular cloning has further revealed the existence of three variants of GlyT1, termed GlyT-la, GlyT-1b and GlyT-1c (Kim et al., Molecular Pharmacology, 45, 1994: 608-617), each of which displays a unique distribution in the brain and peripheral tissues. The variants arise by differential splicing and exon usage, and differ in their N-terminal regions. GlyT2, in contrast, is found predominantly in the brain stem and spinal cord, and its distribution corresponds closely to that of strychnine-sensitive glycine receptors (Liu et al., J. Biological Chemistry, 268, 1993: 22802-22808; Jursky and Nelson, J. Neurochemistry, 64, 1995: 1026-1033). Another distinguishing feature of glycine transport mediated by GlyT2 is that it is not inhibited by sarcosine as is the case for glycine transport mediated by GlyT1. These data are consistent with the view that, by regulating the synaptic levels of glycine, GlyT1 and GlyT2 selectively influence the activity of NMDA receptors and strychnine-sensitive glycine receptors, respectively.

NMDA receptors are critically involved in memory and learning (Rison and Staunton, Neurosci. Biobehav. Rev., 19 533-552 (1995); Danysz et al, Behavioral Pharmacol., 6 455-474 (1995)); and, furthermore, decreased function of NMDA-mediated neurotransmission appears to underlie, or contribute to, the symptoms of schizophrenia (Olney and Farber, Archives General Psychiatry, 52, 998-1007 (1996). Thus, agents that inhibit GlyT1 and thereby increase glycine activation of NMDA receptors can be used as novel antipsychotics and anti-dementia agents, and to treat other diseases in which cognitive processes are impaired, such as attention deficit disorders and organic brain syndromes. Conversely, over-activation of NMDA receptors has been implicated in a number of disease states, in particular the neuronal death associated with stroke and possibly neurodegenerative diseases, such as Alzheimer's disease, multi-infarct dementia, AIDS dementia, Huntington's disease, Parkinson's disease, amyotrophic lateral sclerosis or other conditions in which neuronal cell death occurs, such as stroke or head trauma. Coyle & Puttfarcken, Science, 262, 689-695 (1993); Lipton and Rosenberg, New End. J. of Medicine, 330, 613-622 (1993); Choi, Neuron, 1, 623-634 (1988). Thus, pharmacological agents that increase the activity of GlyT1 will result in decreased glycine-activation of NMDA receptors, which activity can be used to treat these and related disease states. Similarly, drugs that directly block the glycine site of the NMDA receptors can be used to treat these and related disease states.

Glycine transport inhibitors are already known in the art, for example as disclosed in published international patent application WO03/055478 (SmithKline Beecham).

However, there still remains the need to identify further compounds that can inhibit GlyT1 transporters, including those that inhibit GlyT1 transporters selectively over GlyT2 transporters.

It has now been found that a novel class of compounds inhibit GlyT1 transporters and are thus of potential utility in the treatment of certain neurological and neuropsychiatric disorders, including schizophrenia.

Thus, in the first aspect, there is provided a compound of formula (I) or a salt thereof:

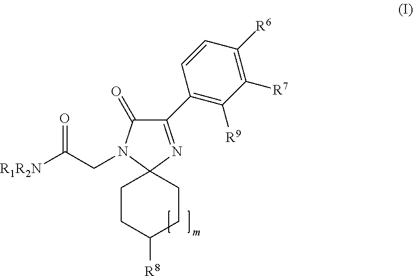

wherein:
R⁶ is selected from haloC₁-C₄alkoxy, haloC₁-C₄alkyl, chloro;
R⁷ is selected from H, C₁-C₄alkyl, C₁-C₄alkoxy, haloC₁-C₄alkyl, haloC₁-C₄alkoxy, halo, cyano, C₁-C₄alkoxyC₁-C₄alkyl and C₁-C₄alkoxyC₁-C₄alkoxy;
R⁸ is selected from hydrogen and methyl;
R⁹ is H or F;
m is 0, 1 or 2;
   and wherein, when R⁶ is haloC₁-C₄alkoxy or haloC₁-C₄alkyl and m is 0, then R¹ is hydrogen and R² is a group —(C₁-C₆alkyl)-Y, wherein Y is a 6-membered carbocyclic saturated or unsaturated ring, optionally substituted by a halogen or a hydroxyl;
   and wherein, when R⁶ is haloC₁-C₄alkoxy or haloC₁-C₄alkyl and m is 1 or 2, then:
R¹ and R² are independently selected from the group consisting of:
   a hydrogen;
   C₁-C₆alkyl;
   C₃-C₆cycloalkyl;
   C₁-C₆alkylC₄-C₆cycloalkyl (wherein the cycloalkyl is optionally substituted by —OH);
   C₃-C₆cycloalkyl fused with phenyl or fused with an unsaturated C₃-C₆carbocyclic group;
   CH₂—CHMe-O-pyridyl; and
   —(CH₂)n-Z wherein n is 1, 2, 3, 4, 5 or 6 and Z is pyrrolyl (optionally substituted by C₁-C₆alkyl), phenyl (substituted by halo, C₁-C₆alkyl or C₁-C₆alkoxy), or thienyl optionally substituted by a C₁-C₆alkyl group;
   with the proviso R¹ and R² are not both H;
or R¹ and R², together with the nitrogen atom to which they are attached, form:
   an azetidinyl, pyrrolidinyl, piperidyl or azepanyl group, each of which being optionally substituted by C₁-C₆alkyl, and each of which is also optionally fused with a 5- or 6 membered aromatic or heteroaromatic group containing one or two heteratoms selected from O, S and N; or
   pyrrolidinyl which is substituted at the 2 position with 2-pyridyl or a 5 membered heteroaromatic group containing one or two heteroatoms selected from O, S and N, or CH₂N(CH₃)₂; or
   piperidyl which is substituted at the 2- or 3-position by a 5- or 6-membered aromatic or heteroaromatic group;
      and wherein, when R⁶ is chloro, m is 0, 1, or 2, then:
R¹ and R² are independently selected from the group consisting of:
   hydrogen;
   C₁-C₆alkyl;
   C₃-C₆cycloalkyl;
   C₄-C₆cycloalkylC₁-C₆alkyl;
   C₃-C₆cycloalkyl fused with phenyl or fused with an unsaturated C₃-C₆-carbocyclic group;
   a group —CH₂—CHMe-O-pyridyl; and
   a group —(CH₂)n-Z wherein n is 1, 2, 3, 4, 5 or 6 and Z is phenyl substituted by a C₁-C₆alkyl or a C₁-C₆alkoxy group, or Z is thienyl substituted by a C₁-C₆alkyl group;
   with the proviso R¹ and R² are not both H;
or R¹ and R², together with the nitrogen atom to which they are attached, form:
   an azetidinyl, pyrrolidinyl, piperidyl or azepanyl group, each of which being optionally substituted by C₁-C₆alkyl, and each of which is also optionally fused with phenyl;
   a pyrrolidinyl which is substituted at the 2 position with a 5 membered heteroaromatic group containing one heteroatom selected from O, S and N; or
   piperidyl which is substituted at the 2- or 3-position by a 5- or 6-membered aromatic or heteroaromatic group containing one or two heteratoms selected from O, S and N.

The notations “C_{x-y}” and “Cₓ-C_{y}” are interchangeable.

As used herein, the term “C₁-C₄alkyl” refers to a straight or branched alkyl group of 1-4 carbon atoms in all isomeric forms. Examples include methyl, ethyl, propyl, isopropyl, butyl, isobutyl, sec-butyl and tert-butyl. As used herein, the term “C₁-C₆alkyl” refers to a straight or branched alkyl group of 1-6 carbon atoms in all isomeric forms. Examples include, but are not limited to, methyl, ethyl, propyl, isopropyl, butyl, isobutyl, sec-butyl, tert-butyl, pentyl and hexyl.

As used herein, the term “C₁-C₄alkoxy” refers to the group —O—C₁-C₄alkyl wherein “C₁-C₄alkyl” is as defined above.

As used herein, the term “C₁-C₄alkoxyC₁-C₄alkyl” refers to the group (C₁-C₄alkyl)-O—(C₁-C₄alkyl), wherein “C₁-C₄alkyl” is as defined above.

As used herein, the term “C₁-C₄alkoxyC₁-C₄alkyoxy” refers to the group —O—(C₁-C₄alkyl)-O C₁-C₄alkyl), wherein “C₁-C₄alkyl” is as defined above.

As used herein, the term “C₃-C₆cycloalkyl” refers to a cycloalkyl group consisting of from 3 to 6 carbon atoms, for example cyclopropane, cyclobutane, cyclopentane or cyclohexane.

As used herein the term “C₄-C₆cycloalkyl-C₁-C₆alkyl” refers to a cycloalkyl group consisting of from 4-6 carbon atoms linked to an alkyl group of 1-6 carbon atoms.

As used herein, the terms “halogen” and its abbreviation “halo” refer to fluorine, chlorine, bromine, or iodine.

As used herein, the term “haloC₁-C₄alkyl” refers to a C₁-C₄alkyl group as defined above which is substituted with any number of fluorine, chlorine, bromine, or iodine atoms, including with mixtures of those atoms. A haloC₁-C₄alkyl group may, for example contain 1, 2 or 3 halogen atoms. For example, a haloC₁-C₄alkyl group may have all hydrogen atoms replaced with halogen atoms. Examples of “haloC₁-C₄alkyl” groups include, but are not limited to, fluoromethyl, difluoromethyl and trifluoromethyl.

As used herein, the term “haloC₁-C₄alkoxy” refers to a C₁-C₄alkoxy group, as defined above, which is substituted with any number of fluorine, chlorine, bromine, or iodine atoms, including with mixtures of those atoms. A haloC₁-C₄alkoxy group may, for example contain 1, 2 or 3 halogen atoms. For example, a haloC₁-C₄alkoxy group may have all hydrogen atoms replaced with halogen atoms. Examples of “haloC₁-C₄alkoxy” groups include, but are not limited to, fluoromethyloxy, difluoromethyloxy and trifluoromethyloxy.

As used herein the term “cyano” refers to a group —CN.

As used herein the term “hydroxyl” refers to a group —OH.

As used herein, the term “C₁-C₄alkylsulfonyl” refers to a group —SO₂(C₁-C₄alkyl). An example is —SO₂CH₃.

As used herein, the term “C₁-C₄alkylthio” refers to a group —S—(C₁-C₄alkyl). An example is —SCH₃.

In one embodiment, R⁶ is selected from chloro, haloC₁-C₂alkyloxy and haloC₁-C₂alkyl. In a further embodiment, R⁶ is selected from trifluoromethoxy, trifluoromethyl and chloro.

In one embodiment, R⁷ is hydrogen.

In one embodiment, m is 0 or 1.

In one embodiment, m is 0.

In one embodiment, m is 1.

In one embodiment, R⁸ is hydrogen.

In one embodiment, when R⁶ is haloC₁-C₄alkoxy or haloC₁-C₄alkyl and m is 0, then R¹ is hydrogen and R² is a group —CH₂—Y wherein Y is a 6-membered carbocyclic saturated or unsaturated ring, substituted by a halogen or a hydroxyl group.

In a further embodiment, when R⁶ is haloC₁-C₄alkoxy or haloC₁-C₄alkyl and m is 0, then R¹ is hydrogen and R² is a group —CH₂—Y wherein Y is 1-hydroxy-cyclohexyl or 2 chlorophenyl.

In one embodiment, when R⁶ is haloC₁-C₄alkoxy or haloC₁-C₄alkyl and m is 1 or 2, then:
R₁ and R₂ are independently selected from the group consisting of:
   Hydrogen;
   C₁-C₅alkyl;
   C₄-C₅cycloalkyl;
   —CH₂CHMeO-pyridinyl;
   —CH₂-thienyl;
   2-methylphenylmethyl; and
   —CH₂CH₂-pyrrolyl (optionally N-substituted by methyl);
   provided that R₁ and R₂ are not both H;
or R₁ and R₂, together with the nitrogen to which they are attached form:
   4,5-dehydropiperidinyl;
   piperidinyl substituted by pyridinyl or dimethylaminomethyl;
   pyrrolidinyl substituted by furyl, thiazolyl, or pyridyl; or
   isoindolinyl.

In a further embodiment, when R⁶ is haloC₁-C₄alkoxy or haloC₁-C₄alkyl and m is 1 or 2, then
R₁ and R₂ are independently selected from the group consisting of hydrogen, methyl, n-butyl, t-butyl, 1-methylpropyl, 1-methylbutyl, 3-methylbutyl, cyclobutyl, 3,4-dehydrocyclopentyl, —CH₂CHMeO-pyridin-3-yl, —CH₂CHMeO-pyridin-2-yl, —CH₂-thien-2-yl, 2-methylphenylmethyl, —CH₂CH₂-pyrrol-2-yl, and —CH₂CH₂-1-methyl-pyrrol-2-yl; provided that R₁ and R₂ are not both H; or
R₁ and R₂, together with the nitrogen to which they are attached, form:
   4,5-dehydropiperidinyl;
   piperidinyl substituted at the 2-position by pyridin-2-yl, pyridin-3-yl or pyridin-4-yl, or substituted at the 3-position by pyridin-2-yl or dimethylaminomethyl; or
   pyrrolidinyl substituted at the 2-position furan-2-yl, thiazol-2-yl, or pyridin-2-yl; or
   isoindolinyl.

In one embodiment, when R⁶ is chloro, m is 0, 1, or 2, then:
R¹ and R² are independently selected from the group consisting of:
   hydrogen, C₄-C₆alkyl, C₄-C₆cycloalkyl, C₁-C₆alkylC₄-C₆cycloalkyl, C₃-C₆cycloalkyl fused with phenyl, and
   a group —CH₂—Z wherein Z is phenyl substituted by a C₁-C₆alkyl or C₁-C₆ alkoxy group, or Z is a thienyl substituted by a C₁-C₆alkyl group;
   with the proviso R¹ and R² are not both H;
or R¹ and R², together with the nitrogen atom to which they are attached, form C₁-C₆alkyl-substituted dihydroisoquinolinyl, or dihydroisoindolyl;
or R¹ and R², together with the nitrogen atom to which they are attached, form:
   a pyrrolidinyl which is substituted at the 2-position with furyl or thienyl, or
   piperidyl which is substituted at the 3-position by pyridine or phenyl.

In a further embodiment, when R⁶ is chloro, m is 0, 1 or 2, then:
R¹ and R² are independently selected from the group consisting of:
   hydrogen, 1-methylbutyl, 3-methylbutyl, n-butyl, t-butyl, 1-methylpropyl, cyclobutyl, cyclopentyl, cyclohexyl, —CH₂-cyclohexyl, 2,3-dihydro-1H-inden-2-yl; and
   a group —CH₂—Z wherein Z is 2-methylphenyl, 2-methoxyphenyl, or 3-methyl-2-thienyl;
   with the proviso R¹ and R² are not both H;
or R¹ and R², together with the nitrogen atom to which they are attached, form 3-methyl-3,4-dihydro-2(1H)-isoquinolin-2-yl, or 1,3-dihydro-2H-isoindol-2-yl;
or R¹ and R², together with the nitrogen atom to which they are attached, form:
   a pyrrolidinyl which is substituted at the 2 position with 2-furyl or 2-thienyl, or
   a piperidyl which is substituted at the 3-position by 2-pyridine or phenyl.

In one embodiment there is provided a compound of formula (Ia) or a salt or solvate thereof:

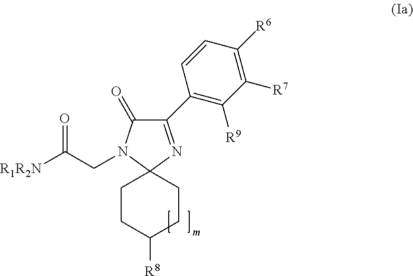

wherein:
R⁶ is selected from haloC₁-C₄alkoxy, haloC₁-C₄alkyl, chloro;
R⁷ is selected from H, C₁-C₄alkyl, C₁-C₄alkoxy, haloC₁-C₄alkyl, haloC₁-C₄alkoxy, halo, cyano, C₁₋₄alkoxyC₁₋₄alkyl and C₁-C₄alkoxyC₁-C₄alkoxy;
R⁸ is selected from hydrogen and methyl; and
R⁹ is H or F,
   when R⁶ is haloC₁-C₄alkoxy or haloC₁-C₄alkyl and m is 0, then R¹ is hydrogen and R² is a group —(C₁₋₆alkyl)-Y wherein Y is a 6-membered carbocyclic saturated or unsaturated ring, optionally substituted by a halogen or a hydroxyl, R⁷, R⁸ and R⁹ are as defined above;
   when R⁶ is haloC₁-C₄alkoxy or haloC₁-C₄alkyl and m is 1 or 2, then:
R¹ and R² are independently selected from the group consisting of hydrogen, C₁₋₆alkyl, C₃₋₆cycloalkyl, C₁₋₆alkylC₄₋₆cycloalkyl (wherein the cycloalkyl is optionally substituted by —OH) or a C₃₋₆cycloalkyl fused with phenyl or an unsaturated C₃₋₆-carbocyclic group with the proviso R¹ and R² are not both H;
or R¹ and R², together with the nitrogen atom to which they are attached, form an azetidinyl, pyrrolidinyl, piperidyl or azepanyl group, each of which being optionally substituted by C₁₋₆alkyl, and each of which is also optionally fused with a 5- or 6 membered aromatic or heteroaromatic group containing one or two heteratoms selected from O, S and N;
or R¹ and R², together with the nitrogen atom to which they are attached, form
pyrrolidinyl which is substituted at the 2 position with 2-pyridyl or a 5 membered heteroaromatic group containing one or two heteroatoms selected from O, S and N, or CH₂N(CH₃)₂ or
piperidyl which is substituted at the 2- or 3-position by a 5- or 6-membered aromatic or heteroaromatic group;
a group pyridyl-O—CHMe-CH₂;
a group Z—(CH₂)n- wherein n is 1, 2, 3, 4, 5 or 6 and Z is pyrrolyl optionally substituted by a C₁₋₆alkyl, or phenyl substituted by a halo, C₁₋₆alkyl or a C₁₋₆alkoxy group, or is a thienyl optionally substituted by a C₁₋₆alkyl group;
R⁷, R⁸ and R⁹ are as defined above;
   when R⁶ is chloro, m is 0, 1, or 2, then:
R¹ and R² are independently selected from the group consisting of hydrogen, C₁₋₆alkyl, C₃₋₆cycloalkyl, C₄₋₆cycloalkylC₁₋₆alkyl or a C₃₋₆cycloalkyl fused with phenyl or an unsaturated C₃₋₆-carbocyclic group with the proviso R¹ and R² are not both H;
or R¹ and R², together with the nitrogen atom to which they are attached, form an azetidinyl, pyrrolidinyl, piperidyl or azepanyl group, each of which being optionally substituted by C₁₋₆alkyl, and each of which is also optionally fused with phenyl;
or R¹ and R², together with the nitrogen atom to which they are attached, form a pyrrolidinyl which is substituted at the 2 position with a 5 membered heteroaromatic group containing one heteroatom selected from O, S and N, or piperidyl which is substituted at the 2-position by a 5- or 6-membered aromatic or heteroaromatic group containing one or two heteratoms selected from O, S and N;
a group pyridyl-O—CHMe-CH2;
a group Z—(CH₂)n- wherein n is 1, 2, 3, 4, 5 or 6 and Z is phenyl substituted by a C₁₋₆alkyl or a C₁₋₆alkoxy group, or Z is a thienyl substituted by a C₁₋₆alkyl group.

R⁷, R⁸ and R⁹ are as defined above.

For the avoidance of doubt, the embodiments of any one feature of the compounds of the invention may be combined with any embodiment of another feature of compounds of the invention to create a further embodiment.

Examples of compounds of the invention include:
N-(1-Hydroxy-cyclohexylmethyl)-2-[2-oxo-3-(4-trifluoromethyl-phenyl)-1,4-diaza-spiro[4.4]non-3-en-1-yl]-acetamide;
N-(2-Chloro-benzyl)-2-[2-oxo-3-(4-trifluoromethyl-phenyl)-1,4-diaza-spiro[4.4]non-3-en-1-yl]-acetamide;
2-{2-oxo-3-[4-(trifluoromethyl)phenyl]-1,4-diazaspiro[4.5]dec-3-en-1-yl}-N-(2-thienylmethyl)acetamide;
1-[2-(3,6-dihydro-1(2H)-pyridinyl)-2-oxoethyl]-3-[4-(trifluoromethyl)phenyl]-1,4-diazaspiro[4.5]dec-3-en-2-one;
N-(1-methylbutyl)-2-{2-oxo-3-[4-(trifluoromethyl)phenyl]-1,4-diazaspiro[4.5]dec-3-en-1-yl}acetamide;
N-(3-methylbutyl)-2-{2-oxo-3-[4-(trifluoromethyl)phenyl]-1,4-diazaspiro[4.5]dec-3-en-1-yl}acetamide;
N-3-cyclopenten-1-yl-2-{2-oxo-3-[4-(trifluoromethyl)phenyl]-1,4-diazaspiro[4.5]dec-3-en-1-yl}acetamide;
N-cyclobutyl-2-{2-oxo-3-[4-(trifluoromethyl)phenyl]-1,4-diazaspiro[4.5]dec-3-en-1-yl}acetamide;
1-{2-oxo-2-[2-(2-pyridinyl)-1-piperidinyl]ethyl}-3-[4-(trifluoromethyl)phenyl]-1,4-diazaspiro[4.5]dec-3-en-2-one;
1-{2-[2-(2-furanyl)-1-pyrrolidinyl]-2-oxoethyl}-3-[4-(trifluoromethyl)phenyl]-1,4-diazaspiro[4.5]dec-3-en-2-one;
N-methyl-N-[2-(1-methyl-1H-pyrrol-2-yl)ethyl]-2-{2-oxo-3-[4-(trifluoromethyl)phenyl]-1,4-diazaspiro[4.5]dec-3-en-1-yl}acetamide;
2-{2-oxo-3-[4-(trifluoromethyl)phenyl]-1,4-diazaspiro[4.5]dec-3-en-1-yl}-N-[2-(3-pyridinyloxy)propyl]acetamide;
1-{2-oxo-2-[2-(4-pyridinyl)-1-piperidinyl]ethyl}-3-[4-(trifluoromethyl)phenyl]-1,4-diazaspiro[4.5]dec-3-en-2-one;
2-[3-(4-chlorophenyl)-2-oxo-1,4-diazaspiro[4.5]dec-3-en-1-yl]-N-(1-methylbutyl)acetamide;
2-[3-(4-chlorophenyl)-2-oxo-1,4-diazaspiro[4.5]dec-3-en-1-yl]-N-{[2-(methyloxy)phenyl]methyl}acetamide;
2-[3-(4-chlorophenyl)-2-oxo-1,4-diazaspiro[4.5]dec-3-en-1-yl]-N-(3-methylbutyl)acetamide;
3-(4-chlorophenyl)-1-{2-oxo-2-[3-(2-pyridinyl)-1-piperidinyl]ethyl}-1,4-diazaspiro[4.5]dec-3-en-2-one;
2-[3-(4-chlorophenyl)-2-oxo-1,4-diazaspiro[4.5]dec-3-en-1-yl]-N-cyclobutylacetamide;
2-[3-(4-chlorophenyl)-2-oxo-1,4-diazaspiro[4.5]dec-3-en-1-yl]-N-[(3-methyl-2-thienyl)methyl]acetamide;
3-(4-chlorophenyl)-1-{2-[2-(2-furanyl)-1-pyrrolidinyl]-2-oxoethyl}-1,4-diazaspiro[4.5]dec-3-en-2-one;
3-(4-chlorophenyl)-1-{2-oxo-2-[2-(2-thienyl)-1-pyrrolidinyl]ethyl}-1,4-diazaspiro[4.5]dec-3-en-2-one;
2-[3-(4-chlorophenyl)-2-oxo-1,4-diazaspiro[4.5]dec-3-en-1-yl]-N-(2,3-dihydro-1H-inden-2-yl)acetamide;
3-(4-chlorophenyl)-1-[2-(3-methyl-3,4-dihydro-2(1H)-isoquinolinyl)-2-oxoethyl]-1,4-diazaspiro[4.5]dec-3-en-2-one;
1-{2-oxo-2-[3-(2-pyridinyl)-1-piperidinyl]ethyl}-3-{4-[(trifluoromethyl)oxy]phenyl}-1,4-diazaspiro[4.5]dec-3-en-2-one;
2-[3-(4-chlorophenyl)-2-oxo-1,4-diazaspiro[4.5]dec-3-en-1-yl]-N-cyclohexylacetamide;
2-[3-(4-chlorophenyl)-2-oxo-1,4-diazaspiro[4.5]dec-3-en-1-yl]-N-cyclopentylacetamide;
1-{2-oxo-2-[3-(2-pyridinyl)-1-piperidinyl]ethyl}-3-[4-(trifluoromethyl)phenyl]-1,4-diazaspiro[4.5]dec-3-en-2-one;
1-{2-oxo-2-[2-(1,3-thiazol-2-yl)-1-pyrrolidinyl]ethyl}-3-[4-(trifluoromethyl)phenyl]-1,4-diazaspiro[4.5]dec-3-en-2-one;
N-butyl-2-{2-oxo-3-[4-(trifluoromethyl)phenyl]-1,4-diazaspiro[4.5]dec-3-en-1-yl}acetamide;
N-(1,1-dimethylethyl)-2-{2-oxo-3-[4-(trifluoromethyl)phenyl]-1,4-diazaspiro[4.5]dec-3-en-1-yl}acetamide;
N-(1-methylpropyl)-2-{2-oxo-3-[4-(trifluoromethyl)phenyl]-1,4-diazaspiro[4.5]dec-3-en-1-yl}acetamide;
N-butyl-2-[3-(4-chlorophenyl)-2-oxo-1,4-diazaspiro[4.5]dec-3-en-1-yl]acetamide;
2-[3-(4-chlorophenyl)-2-oxo-1,4-diazaspiro[4.5]dec-3-en-1-yl]-N-(1,1-dimethylethyl)acetamide;
2-[3-(4-chlorophenyl)-2-oxo-1,4-diazaspiro[4.5]dec-3-en-1-yl]-N-(1-methylpropyl)acetamide;
N-[2-(1-methyl-1H-pyrrol-2-yl)ethyl]-2-{2-oxo-3-[4-(trifluoromethyl)phenyl]-1,4-diazaspiro[4.5]dec-3-en-1-yl}acetamide;
2-{2-oxo-3-[4-(trifluoromethyl)phenyl]-1,4-diazaspiro[4.5]dec-3-en-1-yl}-N-[2-(2-pyridinyloxy)propyl]acetamide;
1-(2-{3-[(dimethylamino)methyl]-1-piperidinyl}-2-oxoethyl)-3-[4-(trifluoromethyl)phenyl]-1,4-diazaspiro[4.5]dec-3-en-2-one;
3-(4-chlorophenyl)-1-[2-oxo-2-(3-phenyl-1-piperidinyl)ethyl]-1,4-diazaspiro[4.5]dec-3-en-2-one;
1-{2-oxo-2-[2-(2-pyridinyl)-1-pyrrolidinyl]ethyl}-3-[4-(trifluoromethyl)phenyl]-1,4-diazaspiro[4.5]dec-3-en-2-one;
1-{2-oxo-2-[2-(3-pyridinyl)-1-piperidinyl]ethyl}-3-[4-(trifluoromethyl)phenyl]-1,4-diazaspiro[4.5]dec-3-en-2-one;
N-[(2-methylphenyl)methyl]-2-{2-oxo-3-[4-(trifluoromethyl)phenyl]-1,4-diazaspiro[4.5]dec-3-en-1-yl}acetamide;
1-[2-(1,3-dihydro-2H-isoindol-2-yl)-2-oxoethyl]-3-[4-(trifluoromethyl)phenyl]-1,4-diazaspiro[4.5]dec-3-en-2-one;
2-[3-(4-chlorophenyl)-2-oxo-1,4-diazaspiro[4.5]dec-3-en-1-yl]-N-[(2-methylphenyl)methyl]acetamide;
2-[3-(4-chlorophenyl)-2-oxo-1,4-diazaspiro[4.5]dec-3-en-1-yl]-N-(cyclohexylmethyl)acetamide; and
3-(4-chlorophenyl)-1-[2-(1,3-dihydro-2H-isoindol-2-yl)-2-oxoethyl]-1,4-diazaspiro[4.5]dec-3-en-2-one; and salts and solvates thereof.

In a further aspect of the present invention, there is provided a pharmaceutical composition comprising a compound of formula (I) or a salt thereof and one or more of pharmaceutically acceptable carriers, diluents and excipients.

In a further aspect of the present invention, there is provided a compound of formula (I), or a salt thereof for use in therapy.

In a further aspect of the present invention, there is provided a method of treating disorders mediated by GlyT1 comprising administering a compound of formula (I) or a salt thereof.

In a further aspect of the present invention, there is provided the use of a compound of formula (I), or a salt thereof in the manufacture of a medicament for use in the treatment of disorders mediated by GlyT1.

In a further aspect of the present invention, there is provided a compound of formula (I), or a salt thereof, for the treatment of disorders mediated by GlyT1.

In a further aspect of the present invention, there is provided a combination comprising a compound of formula (I) or a salt thereof and one or more therapeutic agents, such as one or more antipsychotic agents.

In a further aspect of the present invention there is provided a combination comprising a compound of formula (I) or a salt thereof and one or more therapeutic agents such as one or more antipsychotic agents for use in therapy, in particular the treatment of disorders mediated by GlyT1.

In a further aspect of the present invention, there is provided processes for the synthesis of compounds of formula (I) and salts thereof.

As used herein, the term “salt” refers to any salt of a compound according to the present invention prepared from an inorganic or organic acid or base, quaternary ammonium salts and internally formed salts. Pharmaceutically acceptable salts are particularly suitable for medical applications because of their greater aqueous solubility relative to the parent compounds. Such salts must clearly have a pharmaceutically acceptable anion or cation. Suitably pharmaceutically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, (1R)-(−)-10-camphorsulphonic, (1S)-(+)-10-camphorsulphonic, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example naphthalene-1,5-disulphonic, naphthalene-1,3-disulphonic, benzenesulfonic, and p-toluenesulfonic, acids; base addition salts formed with alkali metals and alkaline earth metals and organic bases such as N,N-dibenzylethylenediamine, chloroprocaine, choline, diethanolamine, ethylenediamine, meglumaine (N-methylglucamine), lysine and procaine; and internally formed salts. Salts having a non-pharmaceutically acceptable anion or cation are within the scope of the invention as useful intermediates for the preparation of pharmaceutically acceptable salts and/or for use in non-therapeutic, for example, in vitro, situations. The salts may have any suitable stoichiometry. For example, a salt may have 1:1 or 2:1 stoichiometry. Non-integral stoichiometry ratios are also possible.

In an embodiment, there is provided a compound of formula (I) as defined above or a pharmaceutically acceptable salt thereof.

Solvates of the compounds of formula (I) and solvates of salts of the compounds of formula (I) are included within the scope of the present invention. As used herein, the term “solvate” refers to a complex of variable stoichiometry formed by a solute (in this invention, a compound of formula (I) or a salt thereof) and a solvent. Those skilled in the art of organic chemistry will appreciate that many organic compounds can form such complexes with solvents in which they are reacted or from which they are precipitated or crystallised. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water. Where the solvent used is water such a solvate may then also be referred to as a hydrate.

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of the invention may be administered as prodrugs. Examples of pro-drug forms for certain compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Examples of prodrugs for certain compounds of the invention include: esters, carbonate esters, hemi-esters, phosphate esters, nitro esters, sulfate esters, sulfoxides, amides, carbamates, azo-compounds, phosphamides, glycosides, ethers, acetals and ketals.

Hereinafter, compounds of formula (I) (whether in solvated or unsolvated form) or their pharmaceutically acceptable salts (whether in solvated or unsolvated form) or prodrugs thereof defined in any aspect of the invention (except intermediate compounds in chemical processes) are referred to as “compounds of the invention”.

The compounds of formula (I) may have the ability to crystallise in more than one form. This is a characteristic known as polymorphism, and it is understood that such polymorphic forms (“polymorphs”) are within the scope of formula (I). Polymorphism generally can occur as a response to changes in temperature or pressure or both and can also result from variations in the crystallisation process. Polymorphs can be distinguished by various physical characteristics known in the art such as x-ray diffraction patterns, solubility, and melting point.

Certain of the compounds described herein may exist in stereoisomeric forms (i.e. they may contain one or more asymmetric carbon atoms), for example when R⁸ in formula (I) is not H. The individual stereoisomers (enantiomers and diastereoisomers) and mixtures of these are included within the scope of the present invention. Stereoisomers may be separated by high-performance liquid chromatography or other appropriate means. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994). Likewise, it is understood that compounds of formula (I) may exist in tautomeric forms other than that shown in the formula and these are also included within the scope of the present invention.

In one embodiment, an optically pure enantiomer of a compound of the present invention is provided. The term “optically pure enantiomer” means that the compound contains greater than about 90% of the desired isomer by weight, such as greater than about 95% of the desired isomer by weight, or greater than about 99% of the desired isomer by weight, said weight percent based upon the total weight of the isomer(s) of the compound.

Compounds of general formula (I) may be prepared by methods known in the art of organic synthesis as set forth in part by the following synthesis schemes. It is also recognised that in all of the schemes described below, it is well understood that protecting groups for sensitive or reactive groups are employed where necessary in accordance with general principles of chemistry. Protecting groups are manipulated according to standard methods of organic synthesis (T. W. Greene and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I).

Typical reaction routes for the preparation of a compound of formula (I) as hereinbefore defined are shown below.

Compounds of formula (I) can be prepared by reacting a compound of formula (II) with a base, for example sodium hydride, in a suitable inert solvent, for example dimethylformamide, followed by treatment with a compound of formula (III) as shown in Scheme 1.

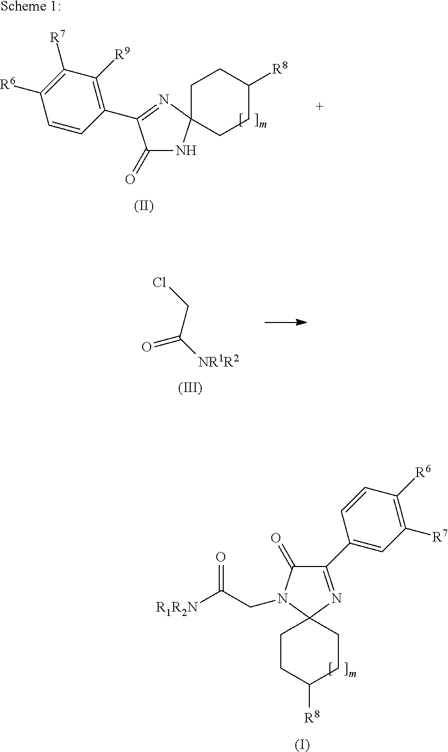

Compounds of formula (III) can be prepared by standard methods, for example as shown in Scheme 2. For example an amine of formula (IV) may be combined with a compound of formula (V) where X and X′ are halogens for example chloroacetyl chloride in an inert solvent, for example dioxan, optionally in the presence of a base such as triethylamine or sodium carbonate to give a compound of formula (III) where X is chloro.

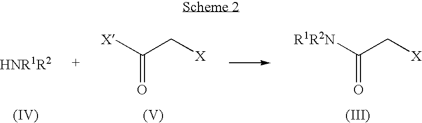

Compounds of formula (II) may be prepared by desulphurisation of compounds of formula (VI) using an oxidising agent, for example hydrogen peroxide as shown for example in Scheme 3.

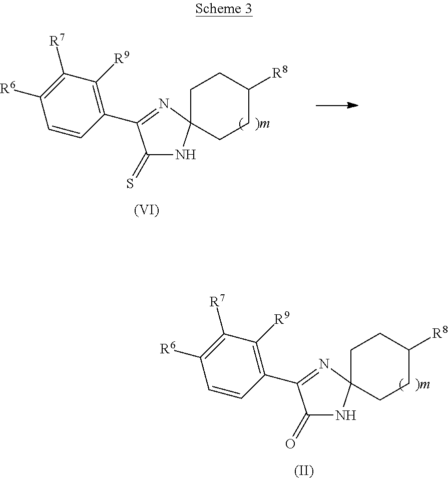

Compounds of formula (VI) can be prepared by treating a ketothioamide of formula (VII) with the appropriate ketone of formula (VIII) in the presence of a source of ammonia, for example ammonium acetate as shown in Scheme 4. Preferably this reaction is performed in a solvent, for example isopropanol, at room or elevated temperature, preferably elevated temperature, for example at reflux.

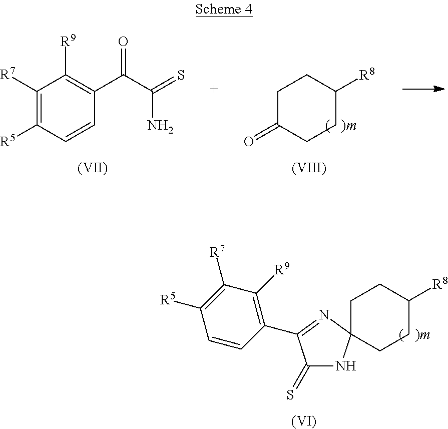

Thioamides of formula (VII) can be prepared from acylnitriles of formula (IX) by treating with, for example hydrogen sulphide in the presence of an organic base, for example triethylamine in an inert solvent, for example diethyl ether at room temperature. Acylnitriles of formula (IX) can be prepared from the appropriate acid chloride (X) and a source of cyanide, conveniently copper (I) cyanide, at elevated temperatures, for example greater than 150° C. preferably in the absence of solvent.

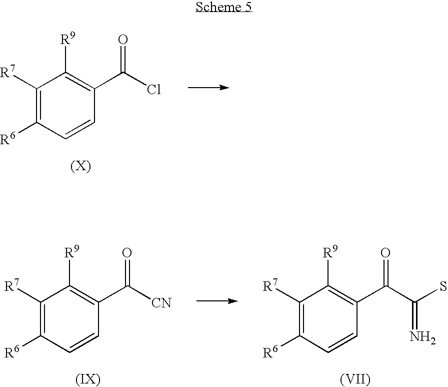

Alternatively, compounds of formula (II) can be synthesised as shown in Scheme 6.

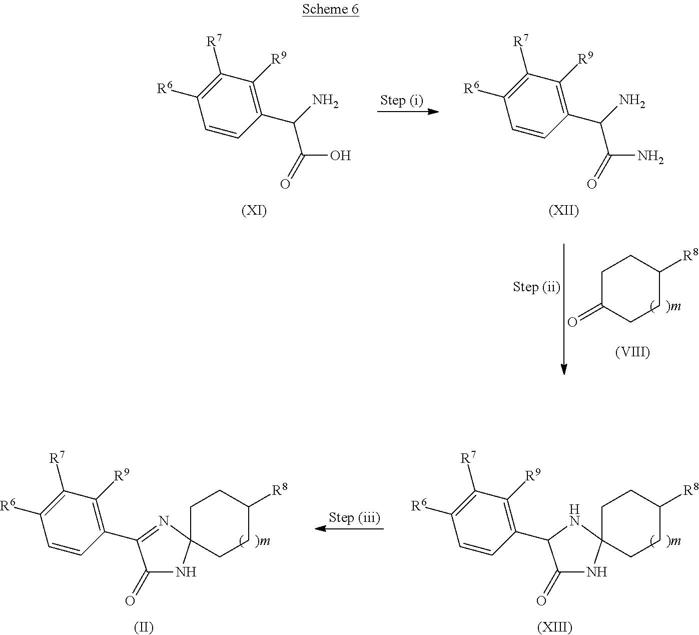

wherein R⁶, R⁷, R⁸ and R⁹ are as defined for formula (I).

The arylglycine of formula (XI) can be converted, step (i), to the corresponding arylglycinamide of formula (XII) by standard methods, for example, by reaction of compounds of formula (XI) with thionyl chloride or acetyl chloride in methanol, followed by subsequent reaction of the intermediate methyl ester hydrochloride with aqueous ammonia.

Arylglycinamides of formula (XII) can be converted to compounds of formula (XIII), step (ii), by condensation with ketones of formula (VIII), for example, by heating in an inert solvent such as methanol, in the presence or absence of a catalyst such as H—Y zeolites.

Oxidation of compounds of formula (XIII), step (iii), to afford compounds of formula (II) can be achieved by methods known in the art, for example, by reaction with N-bromosuccinimide in an inert solvent, such as dichloromethane.

Compounds of formula (II) can also be converted to compounds of formula (I) as shown in Scheme 7.

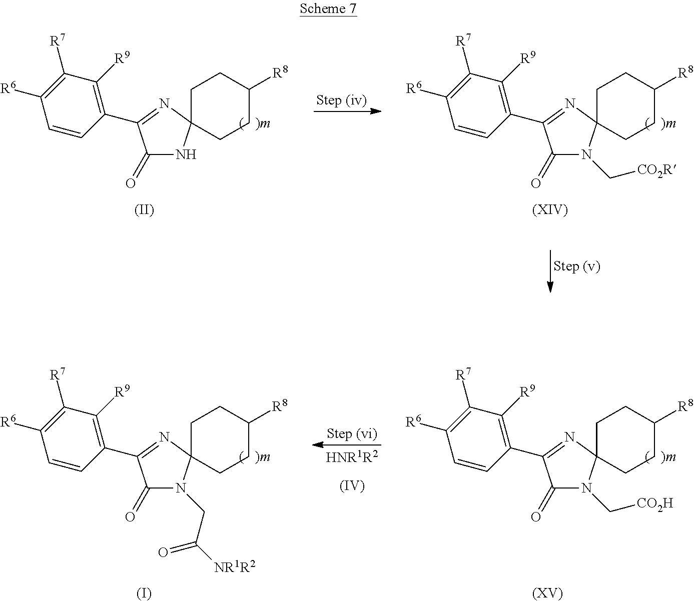

wherein R¹, R², R⁵, R⁶, R⁷, R⁸ and R⁹ are as defined for compounds of formula (I).

Compounds of formula (XIV) can be prepared using standard methods from compounds of formula (II), step (iv), for example, by reaction with an appropriate alkyl haloacetate, for example, ethyl bromoacetate, in the presence of a base, such as sodium hydride or potassium carbonate, in a suitable inert solvent, such as dimethylformamide or acetone, at room temperature or elevated temperature as appropriate.

Removal of the ester group R′ from compounds of formula (XIV) to afford the acids of formula (XV), step (v), can be achieved by known methods, for example by use of a base, such as sodium hydroxide, in an inert solvent, such as aqueous methanol or aqueous ethanol, with or without heating as appropriate.

Compounds of formula (XV) can be converted to compounds of formula (I), step (vi), by reaction with an amine of formula (IV) using a variety of methods known in the art. For example, the acylation step (vi) can be achieved by reaction of the acid (XV) with an amine of formula (IV), in an inert solvent, such as dichloromethane in the presence of a coupling reagent, for example a diimide reagent such as N,N dicyclohexylcarbodiimide (DCC), N-(3-(dimethylamino)propyl)-N-ethylcarbodiimide hydrochloride (EDC), or O-(7-azabenzotriazol-1-yl)-1,1,3,3-tetramethyluronium hexafluoro phosphate (HATU). Alternatively, compounds of formula (XV) can be converted to compounds of formula (XVI).

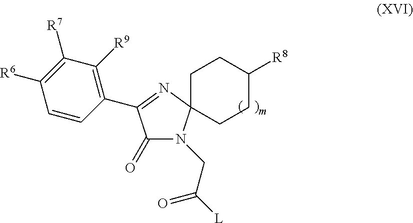

wherein R⁶, R⁷, R⁸ and R⁹ are as defined in formula (I) and L represents a suitable leaving group. Examples of leaving groups include halogen, —OC(═O)alkyl, —OC(═O)O-alkyl and —OSO₂Me. L may be halogen and acylation in step (vi) may be carried out in an inert solvent, such as dichloromethane, in the presence of a base, such as triethylamine.

Within the scheme there is scope to convert a group R¹ into another group R¹ and similarly for groups R², R⁶, R⁶, R⁷ and R⁹.

Compounds of formula (I) can be converted into further compounds of formula (I) using standard techniques. Salts may be prepared conventionally by reaction with the appropriate acid or acid derivative by the following non-limiting examples.

The compounds of the present invention inhibit the GlyT1 transporter as measured by the assay below. Such compounds are therefore of potential utility for the treatment of certain neurological and neuropsychiatric disorders. The compounds may selectively inhibit the GlyT1 transporter over the GlyT2 transporter. Some compounds of the invention may have mixed GlyT1/GlyT2 activity.

The affinities of the compounds of this invention for the GlyT1 transporter can be determined by the following assay. In the assays used herein, the compounds of the present invention were not necessarily from the same batch described above. The test compound made in one batch may have been combined with other batch(es) or the assay(s).

HEK293 cells expressing the Glycine (Type 1) transporter were grown in cell culture medium [DMEM/NUT mix F12 containing 2 mM L-Glutamine, 0.8 mg/mL G418 and 10% heat inactivated fetal calf serum] at 37° C. and 5% CO₂. Cells grown to 70-80% confluency in T175 flasks were harvested and resuspended at 4×10⁵ cells/mL in assay buffer [140 mM NaCl, 5.4 mM KCl, 1.8 mM CaCl₂, 0.8 mM MgSO₄, 20 mM HEPES, 5 mM glucose and 5 mM alanine, pH 7.4]. Compounds were serially diluted 2.5-fold in DMSO from a top concentration of 2.5 mM with each compound giving a 11 data point dose-response. 100 nL of compound at each concentration was added to the assay plate. An equal volume of Leadseeker™ WGA SPA beads (12.5 mg/ml suspended in assay buffer) was added to the cell suspension and 54 of the cell/bead suspension transferred to each well of a 384-well white solid bottom plate (1,000 cells/well) containing 100 nL of test compounds. Substrate (5 μL) was added to each well [1:100 dilution of [³H]-glycine stock in assay buffer containing 2.5 μM glycine). Final DMSO concentration was 1% v/v. Data was collected using a Perkin Elmer Viewlux. pIC₅₀ values were determined using ActivityBase.

Compounds are considered to have activity at the GlyT1 transporter if they have a pIC₅₀ of 5.0 or above, conveniently a pIC₅₀ at the GlyT1 transporter of equal to or greater than 5.9. In one aspect, compounds of the invention have an average pIC₅₀ at the GlyT1 transporter of greater than 7.0.

As used herein, the term “disorders mediated by GlyT1” refers to disorders that may be treated by the administration of a medicament that alters the activity of the GlyT1 transporter. The disorders mediated by GlyT1 referred to herein include neurological and neuropsychiatric disorders, including psychoses such as schizophrenia, dementia and other forms of impaired cognition such as attention deficit disorders and organic brain syndromes. Other neuropsychiatric disorders include drug-induced (phencyclidine, ketamine and other dissociative anesthetics, amphetamine and other psychostimulants and cocaine) psychosis, psychosis associated with affective disorders, brief reactive psychosis, schizoaffective psychosis, and psychosis NOS, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), and NMDA receptor-related disorders such as autism, depression, benign forgetfulness, childhood learning disorders and closed head injury. Other disorders include Parkinson's disease, dyskinetic disorders, cognitive impairment, emesis, movement disorders, amnesia, circadian rhythm disorders, aggression and vertigo.

In one embodiment, the disorder mediated by GlyT1 to be treated by the use or method as hereinbefore described is a psychosis, including schizophrenia, dementia and attention deficit disorders. In one embodiment, the disorder is schizophrenia.

As used herein, the term “effective amount” means that amount of a drug or pharmaceutical agent that will elicit the biological or medical response of a tissue, system, animal or human that is being sought, for instance, by a researcher or clinician.

Within the context of the present invention, the terms used herein are classified in the Diagnostic and Statistical Manual of Mental Disorders, 4^{th} Edition, published by the American Psychiatric Association (DSM-IV) and/or the International Classification of Diseases, 10^{th} Edition (ICD-10). The various subtypes of the disorders mentioned herein are contemplated as part of the present invention. Numbers in brackets after the listed diseases below refer to the classification code in DSM-IV.

In particular, the compounds of the invention may be of use in the treatment of schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

The compounds of the invention may also be of use in the treatment of mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

The compounds of the invention may also be of use in the treatment of anxiety disorders including Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder and Anxiety Disorder Not Otherwise Specified (300.00).

The compounds of the invention may also be of use in the treatment of substance-related disorders including Substance Use Disorders such as Substance Dependence and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

The compounds of the invention may also be of use in the treatment of sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type.

The compounds of the invention may also be of use in the treatment of eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50).

The compounds of the invention may also be of use in the treatment of Autistic Disorder (299.00); Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

The compounds of the invention may also be of use in the treatment of Personality Disorders including the subtypes Paranoid Personality Disorder (301.0), Schizoid Personality Disorder (301.20), Schizotypal Personality Disorder (301.22), Antisocial Personality Disorder (301.7), Borderline Personality Disorder (301.83), Histrionic Personality Disorder (301.50), Narcissistic Personality Disorder (301.81), Avoidant Personality Disorder (301.82), Dependent Personality Disorder (301.6), Obsessive-Compulsive Personality Disorder (301.4) and Personality Disorder Not Otherwise Specified (301.9).

The compounds of the invention may also be of use in the treatment of cognitive impairment. Within the context of the present invention, the term cognitive impairment includes for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypothyroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

The compounds of the present invention are may also be of use for the treatment of cognition impairment which arises in association or as a result of other diseases such as schizophrenia, bipolar disorder, depression, other psychiatric disorders and psychotic conditions associated with cognitive impairment.

The compounds of the invention may also be of use in the treatment of sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

The compounds of the invention may also be of use as anticonvulsants. The compounds of the invention are thus useful in the treatment of convulsions in mammals, and particularly epilepsy in humans. “Epilepsy” is intended to include the following seizures: simple partial seizures, complex partial seizures, secondary generalised seizures, generalised seizures including absence seizures, myoclonic seizures, clonic seizures, tonic seizures, tonic clonic seizures and atonic seizures. The invention also provides a method of treating convulsions, which comprises administering to a mammal in need thereof an effective amount of a compound of formula (I) as hereinbefore described or a salt thereof. Treatment of epilepsy may be carried out by the administration of a non-toxic anticonvulsant effective amount of a compound of the formula (I) or a salt thereof.

The compounds of the invention may also be of use in the treatment of neuropathic pain, for example in diabetic neuropathy, sciatica, non-specific lower back pain, multiple sclerosis pain, fibromyalgia, HIV-related neuropathy, neuralgia such as post-herpetic neuralgia and trigeminal neuralgia and pain resulting from physical trauma, amputation, cancer, toxins or chronic inflammatory conditions.

The compounds of the invention may also be suitable for combination with other therapeutic agents, such as typical and atypical antipsychotics.

As used herein, the terms “treatment” and “treating” refer to the alleviation and/or cure of established symptoms as well as prophylaxis.

The invention thus provides compounds of formula (I) and salts thereof for use in therapy, particularly in the treatment of a disorder mediated by GlyT1.

The invention also provides compounds of formula (I) for use in the treatment of a disorder mediated by GlyT1.

In a further aspect of the present invention, there is provided a method of treating disorders mediated by GlyT1 comprising administering a compound of formula (I) or a salt thereof.

In a further aspect of the present invention there is provided the use of a compound of formula (I) or a salt thereof in the manufacture of a medicament for use in the treatment of disorders mediated by GlyT1.

In a further aspect of the present invention there is provided a compound of formula (I) or a salt thereof for treatment of disorders mediated by GlyT1.

In order to use a compound of the present invention in therapy, it will normally be formulated into a pharmaceutical composition in accordance with standard pharmaceutical practice. The present invention also provides a pharmaceutical composition, which comprises a compound of formula (I) or a salt thereof, and one or more pharmaceutically acceptable carriers, diluents and excipients.

In a further aspect, the present invention provides a process for preparing a pharmaceutical composition, the process comprising mixing a compound of formula (I) or a salt thereof and one or more pharmaceutically acceptable carriers, diluents or excipients.

A pharmaceutical composition of the invention is usually adapted for oral, sub-lingual, buccal, parenteral (for example, subcutaneous, intramuscular, or intravenous), rectal, topical and intranasal administration and in forms suitable for administration by inhalation or insufflation (either through the mouth or nose). The most suitable means of administration for a particular patient will depend on the nature and severity of the conditions being treated and on the nature of the active compound. In one embodiment, oral administration is provided.

Compositions suitable for oral administration may be provided as discrete units, such as tablets, capsules, cachets, or lozenges, each containing a predetermined amount of the active compound; as powders or granules; as solutions or suspensions in aqueous or non-aqueous liquids; or as oil-in-water or water-in-oil emulsions.

Compositions suitable for sublingual or buccal administration include lozenges comprising the active compound and, typically, a flavoured base, such as sugar and acacia or tragacanth and pastilles comprising the active compound in an inert base, such as gelatin and glycerin or sucrose and acacia.

Compositions suitable for parenteral administration typically comprise sterile aqueous solutions containing a predetermined concentration of the active compound; the solution may be isotonic with the blood of the intended recipient. Such solutions may be administered intravenously or by subcutaneous or intramuscular injection.

Compositions suitable for rectal administration may be provided as unit-dose suppositories comprising the active ingredient and one or more solid carriers forming the suppository base, for example, cocoa butter.

Compositions suitable for topical or intranasal application include ointments, creams, lotions, pastes, gels, sprays, aerosols and oils. Suitable carriers for such compositions include petroleum jelly, lanolin, polyethylene glycols, alcohols, and combinations thereof.

The compositions of the invention may be prepared by any suitable method, typically by uniformly and intimately admixing the active compound(s) with liquids or finely divided solid carriers, or both, in the required proportions and then, if necessary, shaping the resulting mixture into the desired shape.

For example, a tablet may be prepared by compressing an intimate mixture comprising a powder or granules of the active ingredient and one or more optional ingredients, such as a binder, lubricant, inert diluent, or surface active dispersing agent, or by moulding an intimate mixture of powdered active ingredient and inert liquid diluent.

Aqueous solutions for parenteral administration are typically prepared by dissolving the active compound in sufficient water to give the desired concentration and then rendering the resulting solution sterile and isotonic.

It will be appreciated that the precise dose administered will depend on the age and condition of the patient and the frequency and route of administration and will be at the ultimate discretion of the attendant physician. The compound may be administered in single or divided doses and may be administered one or more times, for example 1 to 4 times per day.

A proposed dose of the active ingredient for use according to the invention for oral, sub-lingual, parenteral, buccal, rectal, intranasal or topical administration to a human (of approximately 70 kg bodyweight) for the treatment of neurological and neuropsychiatric disorders mediated by a GlyT1 inhibitor, including schizophrenia, may be about 0.1 to about 1000 mg, for example about 0.5 mg to about 1000 mg, or about 1 mg to about 1000 mg, or about 5 mg to about 500 mg, or about 10 mg to about 100 mg of the active ingredient per unit dose, which could be administered, for example, 1 to 4 times per day.

The compounds of formula (I) and their salts thereof may also be suitable for combination with other therapeutic agents, such as typical and atypical antipsychotics. Thus, the present invention also provides:
i) a combination comprising a compound of formula (I) with one or more further therapeutic agents such an one or more antipsychotics;
ii) a pharmaceutical composition comprising a combination product as defined in i) above and at least one carrier, diluent or excipient;
iii) the use of a combination as defined in i) above in the manufacture of a medicament for treating or preventing a disease or condition caused by a reduction or imbalance in glutamate receptor function in a mammal;
iv) a combination as defined in i) above for use in treating or preventing a disease or condition caused by a reduction or imbalance in glutamate receptor function in a mammal;
v) a kit-of-parts for use in the treatment of a psychotic disorder comprising a first dosage form comprising a compound of the invention and one or more further dosage forms each comprising a antipsychotic agent for simultaneous therapeutic administration.
vi) a combination as defined in i) above for use in therapy;
vii) a method of treatment or prevention of a disease or condition caused by a reduction or imbalance in glutamate receptor function in a mammal comprising administering an effective amount of a combination as defined in i) above;
viii) a combination as defined in i) above for the treatment of a disease or condition caused by a reduction or imbalance in glutamate receptor function in a mammal.

The combination therapies of the invention may be administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the compounds of formula (I) or a salt thereof and at least one antipsychotic agent are within the scope of the current invention. In one embodiment of adjunctive therapeutic administration as described herein, a patient is typically stabilised on a therapeutic administration of one or more of the of the components for a period of time and then receives administration of another component. Within the scope of this invention, the compounds of formula (I) or a salt thereof may be administered as adjunctive therapeutic treatment to patients who are receiving administration of at least one antipsychotic agent, but the scope of the invention also includes the adjunctive therapeutic administration of at least one antipsychotic agent to patients who are receiving administration of compounds of formula (I) or a salt thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a further aspect therefore, the invention provides a method of treatment of a psychotic disorder by adjunctive therapeutic administration of compounds of formula (I) or a salt thereof to a patient receiving therapeutic administration of at least one antipsychotic agent. In a further aspect, the invention provides the use of compounds of formula (I) or a salt thereof in the manufacture of a medicament for adjunctive therapeutic administration for the treatment of a psychotic disorder in a patient receiving therapeutic administration of at least one antipsychotic agent. The invention further provides compounds of formula (I) or a salt thereof for use for adjunctive therapeutic administration for the treatment of a psychotic disorder in a patient receiving therapeutic administration of at least one antipsychotic agent.

In a further aspect, the invention provides a method of treatment of a psychotic disorder by adjunctive therapeutic administration of at least one antipsychotic agent to a patient receiving therapeutic administration of compounds of formula (I) or a salt thereof. In a further aspect, the invention provides the use of at least one antipsychotic agent in the manufacture of a medicament for adjunctive therapeutic administration for the treatment of a psychotic disorder in a patient receiving therapeutic administration of compounds of formula (I) or a salt thereof. The invention further provides at least one antipsychotic agent for adjunctive therapeutic administration for the treatment of a psychotic disorder in a patient receiving therapeutic administration of compounds of formula (I) or a salt thereof.

In a further aspect, the invention provides a method of treatment of a psychotic disorder by simultaneous therapeutic administration of compounds of formula (I) or a salt thereof in combination with at least one antipsychotic agent. The invention further provides the use of a combination of compounds of formula (I) or a salt thereof and at least one antipsychotic agent in the manufacture of a medicament for simultaneous therapeutic administration in the treatment of a psychotic disorder. The invention further provides the use of compounds of formula (I) or a salt thereof in the manufacture of a medicament for simultaneous therapeutic administration with at least one antipsychotic agent in the treatment of a psychotic disorder. The invention further provides compounds of formula (I) or a salt thereof for use for simultaneous therapeutic administration with at least one antipsychotic agent in the treatment of a psychotic disorder. The invention further provides the use of at least one antipsychotic agent in the manufacture of a medicament for simultaneous therapeutic administration with compounds of formula (I) or a salt thereof in the treatment of a psychotic disorder.

In further aspects, the invention provides a method of treatment of a psychotic disorder by simultaneous therapeutic administration of a pharmaceutical composition comprising compounds of formula (I) or a salt thereof and at least one mood stabilising or antimanic agent, a pharmaceutical composition comprising compounds of formula (I) or a salt thereof and at least one mood stabilising or antimanic agent, the use of a pharmaceutical composition comprising compounds of formula (I) or a salt thereof and at least one mood stabilising or antimanic agent in the manufacture of a medicament for the treatment of a psychotic disorder, and a pharmaceutical composition comprising compounds of formula (I) or a salt thereof and at least one mood stabilising or antimanic agent for use in the treatment of a psychotic disorder.

Examples of antipsychotic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benzisothiazolyl-piperazines; triazine such as lamotrigine; dibenzoxazepines, such as loxapine; dihydroindolones, such as molindone; aripiprazole; and derivatives thereof that have antipsychotic activity.

Examples of tradenames and suppliers of selected antipsychotic drugs are as follows: clozapine (available under the tradename CLOZARIL®, from Mylan, Zenith Goldline, UDL, Novartis); olanzapine (available under the tradename ZYPREX®, from Lilly; ziprasidone (available under the tradename GEODON®, from Pfizer); risperidone (available under the tradename RISPERDAL®, from Janssen); quetiapine fumarate (available under the tradename SEROQUEL®, from AstraZeneca); haloperidol (available under the tradename HALDOL®, from Ortho-McNeil); chlorpromazine (available under the tradename THORAZINE®, from SmithKline Beecham (GSK)); fluphenazine (available under the tradename PROLIXIN®, from Apothecon, Copley, Schering, Teva, and American Pharmaceutical Partners, Pasadena); thiothixene (available under the tradename NAVANE®, from Pfizer); trifluoperazine (10-[3-(4-methyl-1-piperazinyl)propyl]-2-(trifluoromethyl)phenothiazine dihydrochloride, available under the tradename STELAZINE®, from Smith Klein Beckman); perphenazine (available under the tradename TRILAFON®; from Schering); thioridazine (available under the tradename MELLARIL®; from Novartis, Roxane, HiTech, Teva, and Alpharma); molindone (available under the tradename MOBAN®, from Endo); and loxapine (available under the tradename LOXITANE®; from Watson). Furthermore, benperidol (Glianimon®), perazine (Taxilan®) or melperone (Eunerpan®) may be used. Other antipsychotic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®), prochlorperazine (available under the tradename COMPAZINE®), methotrimeprazine (available under the tradename NOZINAN®), pipotiazine (available under the tradename PIPOTRIL®), ziprasidone, and hoperidone.

It will be appreciated by those skilled in the art that the compounds according to the invention may advantageously be used in conjunction with one or more other therapeutic agents, for instance, antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as cognitive enhancers.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the invention include for example divalproex, carbamazepine and diazepam.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

The invention is further illustrated by the following non-limiting examples.

### Abbreviations:

DCM Dichloromethane
DMF Dimethylformamide
DMSO Dimethylsulphoxide
EDC N-(3-Dimethylaminopropyl-N′-ethylcarbodiimide
HOAT 1-Hydroxyazabenzotriazole
HATU O-(7-Azabenzotriazol-1-yl)-N,N,N′,N′-tetramethyluronium hexafluorophosphate
NMP N-methylpyrrolidin-2-one
MDAP Mass-directed auto-purification
iPrOH Isopropyl alcohol
s.g. Specific gravity
TFA Trifluoroacetic acid
EtOAc Ethyl acetate
g grams
ml millilitres
mmol millimoles

### General Analytical Conditions

### Analytical LC/MS Chromatography Conditions:

Column: Waters Atlantis 50 mm×4.6 mm, 3 μm particle size
Mobile phase: A: 0.05% Formic acid+Water
B: Acetonitrile+0.05% Formic acid
   Gradient: 5-min runtime: 3% B to 97% B over 4 min
   Flow rate: 3 ml/min
   UV wavelength range: 220-330 nm

### Temperature: 30° C.

### Mass Directed Auto-Purification System Chromatography Conditions:

### Method 1

Column: Waters Atlantis 19 mm×100 mm or 30 mm×100 mm, 5 μm particle size
Mobile phase: A: 0.1% Formic acid+Water
   B: Acetonitrile+0.1% Formic acid
Gradient: 13.5 min runtime with 10 min gradient dependant on analytical retention time
Flow rate: 20 or 40 ml/min

### Method 2

Mass-directed HPLC refers to methods where the material was purified by HPLC comprised of the following apparatus: Waters 600 gradient pump, Waters 2767 inject/collector, Waters reagent manager, Micromass ZMD mass spectrometer, Gilson Aspec—waste collector, Gilson 115 post-fraction UV detector. Detection was by UV and fraction collection was triggered by observation of the programmed mass ion for the compound of interest. Software used was Micromass Masslynx version 4.0. The column used was typically a Supelco LCABZ++ column whose dimensions are 20 mm internal diameter by 100 mm in length. The stationary phase particle size was 5 μm. Eluting solvents were: water+0.1% formic acid (solvent A) and acetonitrile:water 95:5+0.05% formic acid (Solvent B). Flow rate of 20 ml/min.

There were five methods used depending on the analytical retention time of the compound of interest. Each has a 15-minute runtime, which comprises of a 10-minute gradient followed by a 5-minute column flush and re-equilibration step:

### MDP 1.5-2.2=0-30%;

### MDP 2.0-2.8=5-30%;

### MDP 2.5-3.0=15-55% B;

### MDP 2.8-4.0=30-80% B;

### MDP 3.8-5.5=50-90% B;

In general, where purifications involve the use of MDAP, Method 1 is employed unless otherwise stated.

¹H NMR spectra were recorded at 294K on a Bruker DPX400 spectrometer using the specified solvent. Chemical shifts are expressed in parts per million (ppm, δ units). Splitting patterns describe apparent multiplicities and are designated as s (singlet), d (doublet), t (triplet), q (quartet), quint. (quintet), sext. (sextet) m (multiplet), br (broad), c (complex).

LCMS spectra were recorded on Waters ZQ spectrometers.

Preparative HPLC refers to methods where the material was purified by High Performance Liquid Chromatography on a Supelcosil ABZ+Plus 5 μm column (10 cm×21.2 mm); Eluting solvents are: water (containing 0.1% TFA) (A) and acetonitrile (containing 0.1% TFA) (B); 10 minute runtime with a gradient elution of 30-85% B at a flow rate of 8 mL/min and UV detection at 254 nm.

### General:

Where reactions are described as having been carried out in a similar manner to earlier, more completely described reactions, the general reaction conditions used were essentially the same. Work up conditions used were of the types standard in the art, but may have been adapted from one reaction to another.

In the procedures that follow, reference to an Intermediate or Example by number is typically provided. This is provided merely for assistance to the skilled chemist to identify the starting material used. The starting material may not necessarily have been prepared from the batch referred to.

### DESCRIPTION 1

### Methyl amino(4-chlorophenyl)acetate

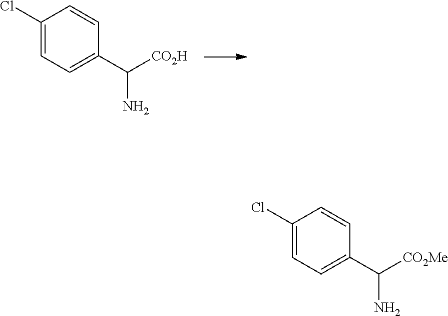

To ice-chilled methanol (300 ml) under argon was carefully added dropwise thionyl chloride (15.44 ml; 0.217 mol) over 45 min. 4-Chlorophenylglycine (26.26 g; 0.141 mol) was added, ice cooling removed and the reaction mixture warmed to 40° C. The reaction was stirred at 40° C. for 48 h. After cooling to room temperature, the reaction was evaporated under reduced pressure. Re-evaporation from methanol afforded a white solid which was triturated with diethyl ether (ca. 700 ml) and then stored at ca. 4° C. for 64 h, filtered, washed with diethyl ether and dried in vacuo to afford the title product as the hydrochloride salt (33.40 g; 100%). ¹H NMR (d₆-DMSO) δ: 3.72 (3H, s), 5.36 (1H, s), 7.53-7.58 (4H, m), 9.07 (3H, s). Mass Spectrum (Electrospray LC/MS). Found 200 (MH⁺). C₉H₁₀³⁵ClNO₂ requires 199. Ret. time 1.32 min.

### DESCRIPTION 2

### 2-Amino-2-(4-chlorophenyl)acetamide

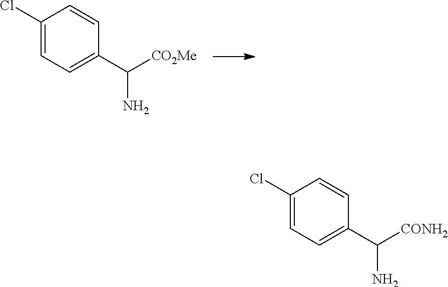

Methyl amino(4-chlorophenyl)acetate D1 as the hydrochloride salt (33.40 g; 0.14 mol) was dissolved in 0.88 ammonia (500 ml; ca. 7.4 mol) and stirred at room temperature for 64 h. The reaction mixture was extracted with DCM (300 ml×6), the extracts dried (Na₂SO₄) and evaporated under reduced pressure to a white solid, which was dried under reduced pressure to afford the title product (22.45 g; 86%). ¹H NMR (CDCl₃) δ: 1.82 (2H, br s), 4.53 (1H, s), 5.49 (1H, br s), 6.92 (1H, br s), 7.32-7.39 (4H, m).

### DESCRIPTION 3

### 3-(4-Chlorophenyl)-1,4-diazaspiro[4.5]decan-2-one

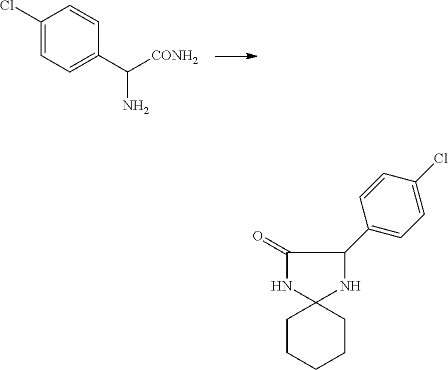

To 2-amino-2-(4-chlorophenyl)acetamide D2 (10.00 g; 54.3 mmol) in methanol (500 ml) was added cyclohexanone (5.62 ml; 54.3 mmol) and H—Y zeolites (10.00 g) and the mixture stirred under reflux for 24 h. The reaction was allowed to cool to room temperature and after 4 days was filtered and the solid washed well with methanol. The filtrate was evaporated to afford the title product (12.91 g; 90%) as a white solid. ¹H NMR (CDCl₃) δ: 1.44-1.57 (4H, m), 1.66-1.76 (6H, m), 2.21 (1H, s), 4.69 (1H, s), 6.80 (1H, s), 7.32-7.35 (2H, m), 7.45-7.49 (2H, m).

### DESCRIPTION 4

### 3-(4-Chlorophenyl)-1,4-diazaspiro[4.5]dec-3-en-2-one

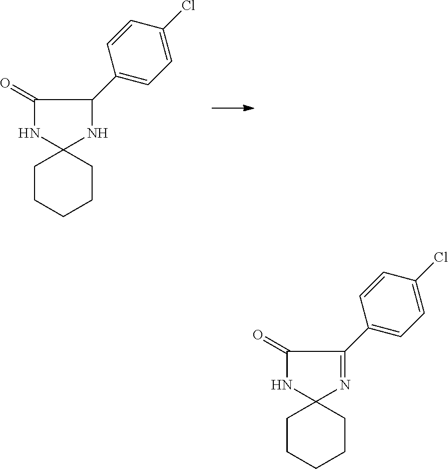

N-Bromosuccinimide (8.69 g; 48.81 mmol) was added in one portion to a stirred solution of 3-(4-chlorophenyl)-1,4-diazaspiro[4.5]decan-2-one D3 (12.91 g; 48.81 mmol) in DCM (400 ml) and the mixture stirred overnight at room temperature. Saturated aqueous sodium bicarbonate (500 ml) was added and the mixture stirred for 0.5 h. The layers were separated and the aqueous extracted with DCM (300 ml). The combined organics were dried (Na₂SO₄) and the solvent removed under reduced pressure at 45° C. The residual solid was partitioned between DCM (500 ml) and saturated aqueous sodium bicarbonate (500 ml) and stirred overnight at room temperature. The aqueous layer was extracted with DCM (300 ml) and the organic layers combined, dried (Na₂SO₄) and the solvent removed under reduced pressure to afford the title product (10.25 g; 80%) as a pale yellow solid. ¹H NMR (CDCl₃) δ: 1.51-1.70 (6H, m), 1.91-1.99 (4H, m), 7.42-7.49 (2H, m), 8.36-8.39 (2H, m), 8.88 (1H, s).

### DESCRIPTION 5

### Ethyl [3-(4-chlorophenyl)-2-oxo-1,4-diazaspiro[4.5]dec-3-en-1-yl]acetate

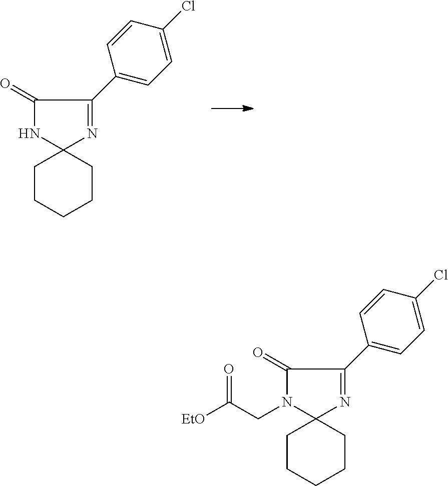

A mixture of 3-(4-chlorophenyl)-1,4-diazaspiro[4.5]dec-3-en-2-one D4 (4.06 g; 15.5 mmol), ethyl bromoacetate (8.55 ml; 77.3 mmol) and potassium carbonate (2.35 g; 17.0 mmol) in acetone (200 ml) was heated at reflux for ca.40 h, cooled and partitioned between water and DCM. The DCM layer was separated, evaporated and the residue chromatographed twice, eluting with ethyl acetate and ethyl acetate-pentane mixtures to afford the title product (3.2 g; 59%). ¹H NMR (CDCl₃) δ: 1.24-1.34 (5H, m), 1.43-1.47 (2H, m), 1.74-1.82 (4H, m), 1.88-1.91 (1H, m), 1.97-2.07 (1H, m), 4.16 (2H, s), 4.20-4.27 (2H, m), 7.41-7.45 (2H, m), 8.43-8.46 (2H, m).

### DESCRIPTION 6

### [3-(4-Chlorophenyl)-2-oxo-1,4-diazaspiro[4.5]dec-3-en-1-yl]acetic acid

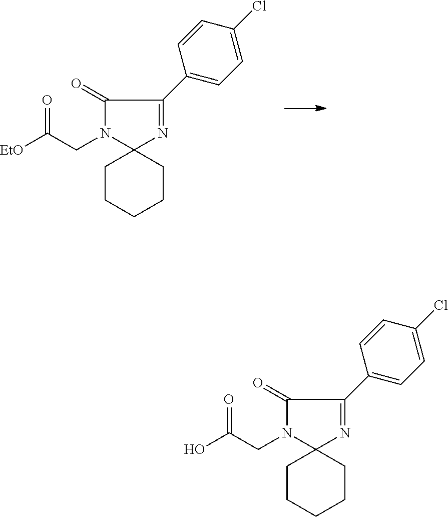

To a stirred mixture of ethyl [3-(4-chlorophenyl)-2-oxo-1,4-diazaspiro[4.5]dec-3-en-1-yl]acetate D5 (4.240 g; 12.17 mmol) in water (150 ml) and methanol (50 ml) was added sodium hydroxide (0.584 g; 14.6 mmol) and the reaction heated at 60° C. overnight, cooled and evaporated under reduced pressure. The residue was partitioned between water (400 ml) and ethyl acetate (200 ml). The aqueous layer was acidified with 5N HCl and extracted into DCM (150 ml×3). The combined DCM extracts were dried (Na₂SO₄) and evaporated under reduced pressure to afford the title acid (3.42 g; 88%) as a colourless solid. ¹H NMR (CDCl₃) δ: 1.25-1.35 (1H, m), 1.44-1.47 (2H, m), 1.76-2.07 (7H, m), 4.22 (2H, s), 7.10-7.70 (1H, br s), 7.41-7.44 (2H, m), 8.39-8.43 (2H, m).

### DESCRIPTION 7

### [3-(4-Chlorophenyl)-2-oxo-1,4-diazaspiro[4.5]dec-3-en-1-yl]acetyl chloride

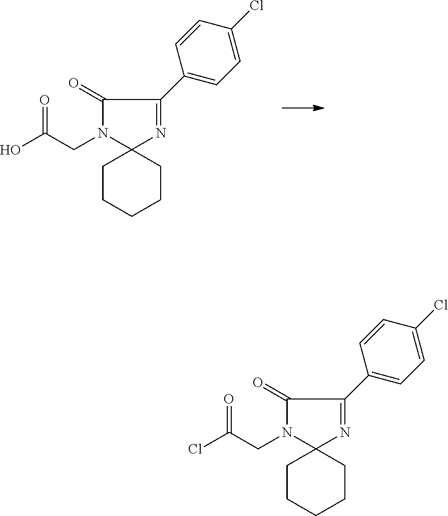

To a suspension of [3-(4-chlorophenyl)-2-oxo-1,4-diazaspiro[4.5]dec-3-en-1-yl]acetic acid D6 (300 mg; 0.936 mmol) in DCM (15 ml) was added oxalyl chloride (0.164 ml; 1.872 mmol) followed with stirring by DMF (1 drop). After stirring overnight the reaction was evaporated under reduced pressure to afford the title product (320 mg; 100%) as a pale yellow solid which was used without further purification. Mass Spectrum (Electrospray LC/MS; MeOH). Found 335 (MH⁺ for methyl ester). C₁₇H₁₉³⁵ClN₂O₃ requires 334. Ret. time 3.5 min.

### DESCRIPTION 8

### Methyl amino[4-(trifluoromethyl)phenyl]acetate

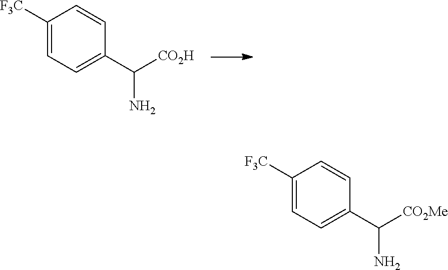

Thionyl chloride (5 ml; 68.9 mmol) was added dropwise under argon to methanol (100 ml) chilled in an ice-bath over 45 min. 4-Trifluoromethylphenylglycine (10 g; 45.6 mmol) was added and the mixture heated at 40° C. for 40 h. After cooling to room temperature, the reaction was evaporated under reduced pressure. The resulting solid was dissolved in methanol (200 ml) and evaporated. Diethylether (250 ml) was added and the product filtered and dried to afford the title compound as the hydrochloride salt (12 g; 98%). ¹H NMR (d₆-DMSO) δ: 3.74 (3H, s), 5.52 (1H, s), 7.74 (2H, d, J=8 Hz), 7.89 (2H, d, J=8 Hz), 9.00 (3H, br s). Mass Spectrum (Electrospray LC/MS) Found 234 (MH⁺). C₁₀H₁₀F₃NO₂ requires 233. Ret. time 1.55 min.

### DESCRIPTION 9

### 2-Amino-2-[4-(trifluoromethyl)phenyl]acetamide

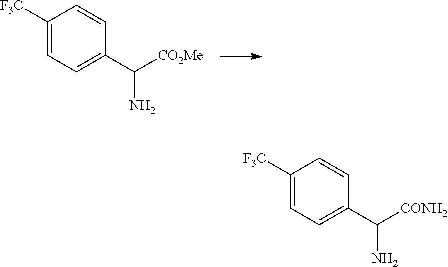

Methyl amino[4-(trifluoromethyl)phenyl]acetate D8 as the hydrochloride salt (12 g; 44.5 mmol) was dissolved in 0.88 ammonia (220 ml; ca. 3.3 mol). After stirring overnight the reaction mixture was extracted with DCM (150 ml×5) and the extracts dried with Na₂SO₄ and the solvent evaporated to afford the title compound (8.92 g; 92%) ¹H NMR (CDCl₃) δ: 1.87 (2H, br s), 4.62 (1H, s), 5.48 (1H, br s), 7.00 (1H, br s) 7.57 (2H, d, J=8 Hz), 7.63 (2H, d, J=8 Hz). Mass Spectrum (Electrospray LC/MS) Found 219 (MH⁺). C₉—H₉F₃N₂O requires 218. Ret. time 1.13 min.

### DESCRIPTION 10

### 3-[4-(Trifluoromethyl)phenyl]-1,4-diazaspiro[4.5]decan-2-one

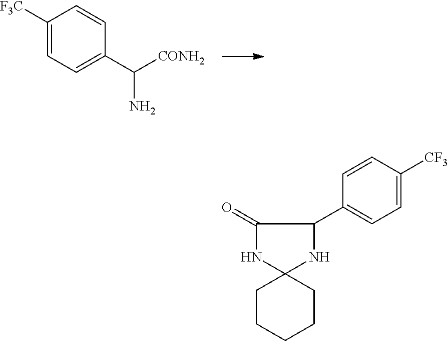

To 2-amino-2-[4-(trifluoromethyl)phenyl]acetamide D9 (8.92 g; 40.9 mmol), in methanol (350 ml) was added cyclohexanone (4.24 ml; 40.9 mmol) and H—Y zeolites (8.92 g) and the mixture refluxed overnight. After cooling to room temperature and chilling in an ice-bath the reaction mixture was filtered. The solid was washed with methanol and the filtrate evaporated under reduced pressure to afford the title compound (10.59 g; 86%). ¹H NMR (CDCl₃) δ: 1.35-1.60 (4H, m), 1.62-1.80 (6H, m), 2.31 (1H, br s), 4.79 (1H, br s) 6.41 (1H, br s), 7.62 (2H, d, J=8 Hz), 7.70 (2H, d, J=8 Hz). Mass Spectrum (Electrospray LC/MS) Found 299 (MH⁺). C₁₅H₁₇F₃N₂O requires 298. Ret. time 2.57 min.

### DESCRIPTION 11

### 3-[4-(Trifluoromethyl)phenyl]-1,4-diazaspiro[4.5]dec-3-en-2-one

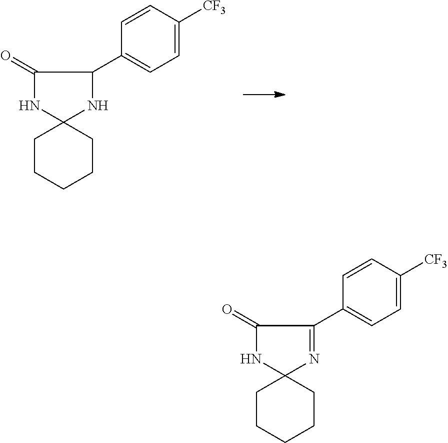

N-Bromosuccinimide (6.32 g; 35.5 mmol) was added to 3-[4-(trifluoromethyl)phenyl]-1,4-diazaspiro[4.5]decan-2-one D10 (10.59 g; 35.5 mmol) in DCM (200 ml) and the reaction stirred overnight at room temperature under argon. Saturated aqueous sodium bicarbonate (150 ml) was added and the mixture stirred, the organic layer was then separated and the aqueous extracted with DCM. The combined DCM extracts were dried with Na₂SO₄, filtered and evaporated under reduced pressure to afford the title product (5 g). Additional washing of the filtered Na₂SO₄ with methanol-DCM afforded further title product, giving 10.69 g in total. Mass Spectrum (Electrospray LC/MS) Found 297 (MH⁺). C₁₅H₁₅F₃N₂O requires 296. Ret. time 3.14 min.

### DESCRIPTION 12

### Ethyl {2-oxo-3-[4-(trifluoromethyl)phenyl]-1,4-diazaspiro[4.5]dec-3-en-1-yl}acetate

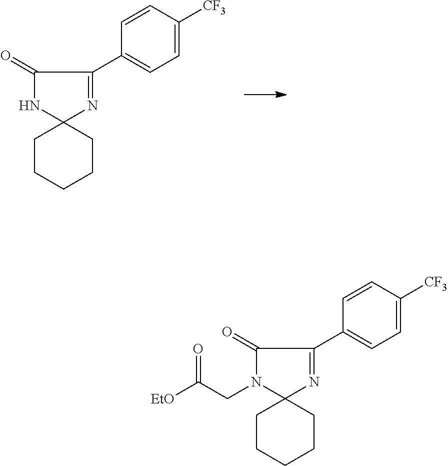

Sodium hydride (0.24 g of a 60% dispersion in oil; 5.95 mmol) was added portionwise over 0.25 h to a stirred solution of 3-[4-(trifluoromethyl)phenyl]-1,4-diazaspiro[4.5]dec-3-en-2-one D11 (1.60 g; 5.41 mmol) in anhydrous DMF (30 ml) at room temperature under an argon atmosphere. After a further 0.25 h ethyl bromoacetate (0.66 ml; 5.95 mmol) was added over 1 minute and the mixture stirred for a further 18 h. Water was added and the mixture extracted with diethyl ether (×4). Combined extracts were washed with water, brine then dried (Na₂SO₄) and the solvent removed under reduced pressure. The crude product was purified by chromatography on silica gel eluting with 0-50% ethyl acetate in pentane gradient to afford the title compound as a pale yellow waxy solid (1.6 g; 77%). ¹H NMR (CDCl₃) δ: 1.20-1.40 (4H, m, including t, J=7.2 Hz), 1.44-1.49 (1H, m), 1.70-2.10 (8H, m), 4.17 (2H, s), 4.23 (2H, q, J=7.2 Hz), 7.71 (2H, d, J=8 Hz), 8.61 (2H, d, J=8 Hz). Mass Spectrum (Electrospray LC/MS) Found 383 (MH⁺). C₁₉H₂₁F₃N₂O₃ requires 382. Ret. time 3.76 min.

### DESCRIPTION 13

### {2-oxo-3-[4-(trifluoromethyl)phenyl]-1,4-diazaspiro[4.5]dec-3-en-1-yl}acetic acid

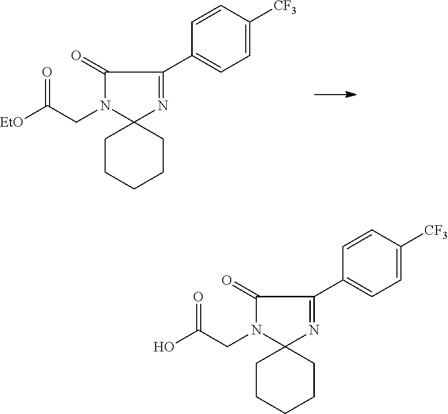

2N Sodium hydroxide (3.18 ml; 6.37 mmol) was added to ethyl {2-oxo-3-[4-(trifluoromethyl)phenyl]-1,4-diazaspiro[4.5]dec-3-en-1-yl}acetate D12 (2.03 g; 5.31 mmol) in methanol (70 ml) and water (30 ml) and the mixture stirred at room temperature overnight. The mixture was evaporated in vacuo and the residue partitioned between water and ethyl acetate. The aqueous layer was acidified to pH1 with 5N HCl and extracted with DCM (×3). The organic extracts were passed through a Biotage Isolute phase separation cartridge and the solvent removed in vacuo to yield the title compound (1.72 g; 91%). Mass Spectrum (Electrospray LC/MS) Found 355 (MH⁺). C₁₇H₁₇F₃N₂O₃ requires 354. Ret. time 3.12 min.

### DESCRIPTION 14

### {2-oxo-3-[4-(trifluoromethyl)phenyl]-1,4-diazaspiro[4.5]dec-3-en-1-yl}acetyl chloride

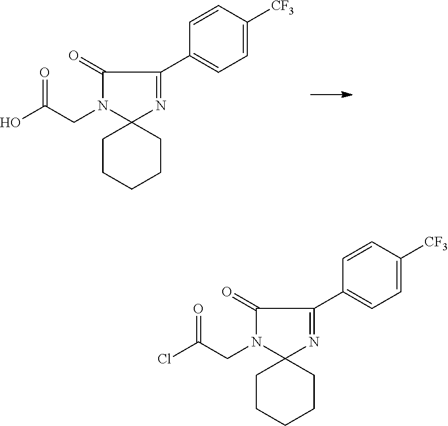

To {2-oxo-3-[4-(trifluoromethyl)phenyl]-1,4-diazaspiro[4.5]dec-3-en-1-yl}acetic acid D13 (1.4 g; 3.95 mmol) in DCM (65 ml) was added oxalyl chloride (0.76 ml; 8.69 mmol) and DMF (3 drops) and the reaction was stirred under argon for 1 h. Evaporation under reduced pressure afforded the title compound (1.46 g; 99%) which was used without further purification. ¹H NMR (CDCl₃) δ: 1.20-1.55 (3H, m), 1.70-2.15 (7H, m), 4.58 (2H, s), 7.72 (2H, d, J=8 Hz), 8.58 (2H, d, J=8 Hz). Mass Spectrum (Electrospray LC/MS; MeOH) Found 369 (MH⁺ for methyl ester). C₁₈H₁₉F₃N₂O₃ requires 368. Ret. time 3.53 min.

### DESCRIPTION 15

### Methyl amino{4-[(trifluoromethyl)oxy]phenyl}acetate

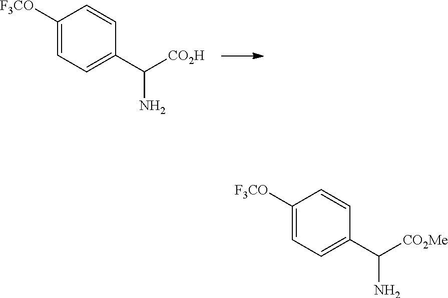

To ice-chilled methanol (30 ml) under argon was carefully added dropwise thionyl chloride (15.44 ml; 0.422 mol) over 30 min. Amino{4-[(trifluoromethyl)oxy]phenyl}acetic acid (5.0 g; 21.280 mmol) was added, ice cooling removed and the reaction mixture was stirred at room temperature for 16 h. The reaction was then evaporated under reduced pressure. Trituration with diethyl ether, followed by filtration provided the title product as the hydrochloride salt, (5.75 g; 95%). ¹H NMR (d₆-DMSO) δ: 3.74 (3H, s), 5.41 (1H, s), 7.51 (2H, d), 7.66 (2H, d), 9.10 (3H, s). Mass Spectrum (Electrospray LC/MS). Found 250 (MH⁺). C₁₀1H₁₀F₃NO₃ requires 249. Ret. time 1.52 min.

### DESCRIPTION 16

### 2-Amino-2-{4-[(trifluoromethyl)oxy]phenyl}acetamide

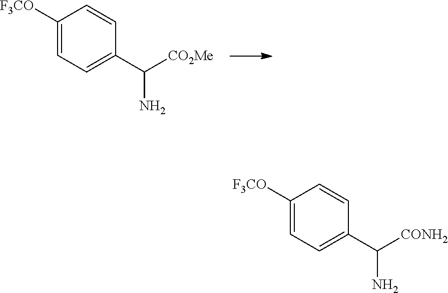

Methyl amino{4-[(trifluoromethyl)oxy]phenyl}acetate D15 as the hydrochloride salt (5.75 g; 20.14 mmol) was dissolved in 0.88 ammonia (75 ml; ca. 1.1 mol) and stirred at room temperature for 16 h. The reaction mixture was extracted with DCM, the extracts dried (MgSO₄) and evaporated under reduced pressure to afford a white solid, which was dried under reduced pressure to afford the title product (3.70 g; 79%). ¹H NMR (d₆-DMSO) δ: 2.22 (2H, br s), 4.32 (1H, s), 7.08 (1H, br s), 7.30 (2H, d), 7.50 (3H, d). Mass Spectrum (Electrospray LC/MS). Found 235 (MH⁺). C₉H₆F₃N₂O₂ requires 234. Ret. time 1.20 min.

### DESCRIPTION 17

### 3-{4-[(Trifluoromethyl)oxy]phenyl}-1,4-diazaspiro[4.5]decan-2-one

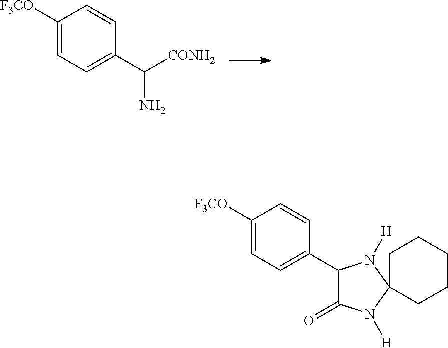

To 2-amino-2-{4-[(trifluoromethyl)oxy]phenyl}acetamide D16 (3.70 g; 15.81 mmol) in methanol (200 ml) was added cyclohexanone (1.549 ml; 15.81 mmol) and H—Y zeolites (6.00 g) and the mixture stirred under reflux for 24 h. The reaction was allowed to cool to room temperature and was filtered and the solid washed well with methanol. The filtrate was evaporated to afford the title product (3.88 g; 50%) as a white solid, after trituration with hexane. ¹H NMR (d₆-DMSO) δ: 1.22-1.45 (2H, m), 1.50-1.70 (8H, m), 3.53 (1H, d), 4.64 (1H, d), 7.32 (2H, d), 7.60 (2H, d), 8.68 (1H, s). Mass Spectrum (Electrospray LC/MS). Found 315 (MH⁺). C₁₅H₁₇F₃N₂O₂ requires 314. Ret. time 2.57 min.

### DESCRIPTION 18

### 3-{4-[(Trifluoromethyl)oxy]phenyl}-1,4-diazaspiro[4.5]dec-3-en-2-one

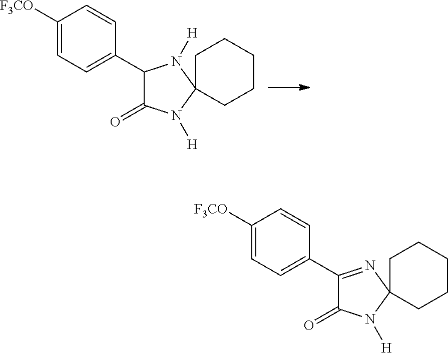

3-{4-[(Trifluoromethyl)oxy]phenyl}-1,4-diazaspiro[4.5]decan-2-one D17 (3.880 g; 12.36 mmol) was dissolved in DCM (80 ml) and stirred at room temperature for 16 hours under an atmosphere of argon with N-bromosuccinimide (2.216 g; 12.36 mmol). A solution of saturated sodium hydrogen carbonate (100 ml) was then added and stirring continued for 1 hour at room temperature. The organic layer was separated, dried (MgSO₄) and evaporated at reduced pressure to yield the title compound as a yellow solid after trituration with hexane (3.25 g; 84%). ¹H NMR (d₆-DMSO) δ: 1.40-1.85 (10H, m), 7.50 (2H, d), 8.47 (2H, d), 10.30 (1H, s). Mass Spectrum (Electrospray LC/MS). Found 313 (MH⁺). C₁₅H₁₅F₃N₂O₂ requires 312. Ret. time 3.23 min.

### DESCRIPTION 19

### Ethyl (2-oxo-3-{4-[(trifluoromethyl)oxy]phenyl}-1,4-diazaspiro[4.5]dec-3-en-1-yl)acetate

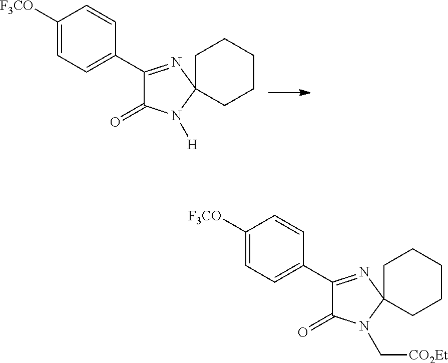

A mixture of 3-{4-[(trifluoromethyl)oxy]phenyl}-1,4-diazaspiro[4.5]dec-3-en-2-one D18 (1.00 g; 3.205 mmol), ethyl bromoacetate (0.354 ml; 3.205 mmol) and potassium carbonate (1.04 g; 7.530 mmol) in DMF (20 ml) was heated at 60° C. for 18 h, with rapid stirring under an atmosphere of argon. After cooling, the reaction solution was poured into water and extracted with ethyl acetate. The organic layer was separated, washed with saturated brine, dried (MgSO₄) and evaporated. The residue was chromatographed over silica gel (50 g), eluting with ethyl acetate-pentane mixtures to afford the title product (0.625 g; 50%) as a colourless oil. ¹H NMR (CDCl₃) δ: 1.24-1.38 (4H, m), 1.41-1.50 (2H, m), 1.74-2.11 (7H, m), 4.18 (2H, s), 4.27 (2H, q), 7.30 (2H, d), 8.57 (2H, d). Mass Spectrum (Electrospray LC/MS). Found 399 (MH⁺). C₁₉H₂₁F₃N₂O₄ requires 398. Ret. time 3.77 min.

### DESCRIPTION 20

### (2-oxo-3-{4-[(trifluoromethyl)oxy]phenyl}-1,4-diazaspiro[4.5]dec-3-en-1-yl)acetic acid

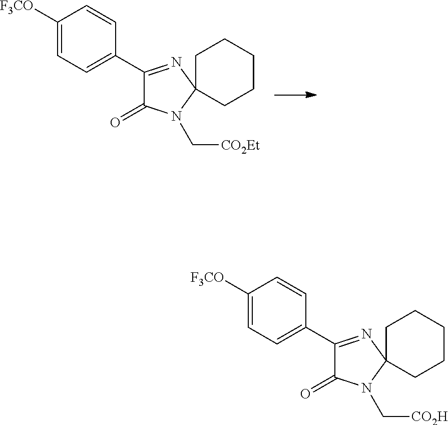

To a stirred mixture of ethyl (2-oxo-3-{4-[(trifluoromethyl)oxy]phenyl}-1,4-diazaspiro[4.5]dec-3-en-1-yl)acetate D19 (0.625 g; 1.57 mmol) in water (30 ml) and methanol (10 ml) was added 2N sodium hydroxide solution (0.95 ml; 1.88 mmol). The reaction solution was heated at 60° C. for 16 hours, cooled and evaporated under reduced pressure. The residue was partitioned between water and ethyl acetate. The aqueous layer was acidified with 5N HCl and extracted into DCM. The DCM extracts were dried (MgSO₄) and evaporated under reduced pressure to afford the title acid (0.513 g; 89%) as a white solid. ¹H NMR (d₆-DMSO) δ: 1.22-1.42 (3H, m), 1.70-1.95 (5H, m), 1.98-2.09 (2H, m), 4.21 (2H, s), 7.58 (2H, d), 8.50 (2H, d), 12.90 (1H, broad s). Mass Spectrum (Electrospray LC/MS). Found 371 (MH⁺). C₁₇H₁₇F₃N₂O₄ requires 370. Ret. time 3.22 min.

### DESCRIPTION 21

### (2-oxo-3-{4-[(trifluoromethyl)oxy]phenyl}-1,4-diazaspiro[4.5]dec-3-en-1-yl)acetyl chloride

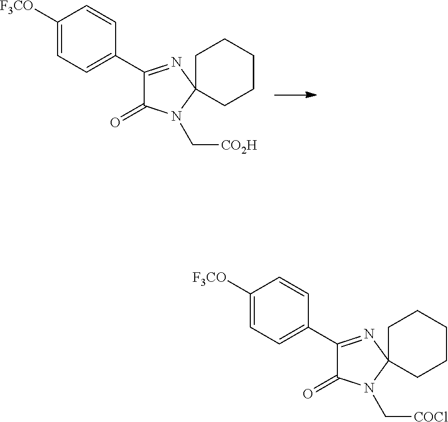

To a suspension of (2-oxo-3-{4-[(trifluoromethyl)oxy]phenyl}-1,4-diazaspiro[4.5]dec-3-en-1-yl)acetic acid D₂O (375 mg; 1.014 mmol) in DCM (15 ml) was added oxalyl chloride (0.205 ml; 2.028 mmol) followed with stirring by DMF (1 drop). After stirring overnight the reaction was evaporated under reduced pressure to afford the title product (396 mg; 100%) as a pale yellow solid which was used without further purification. Mass Spectrum (Electrospray LC/MS; MeOH). Found 385 (MH⁺ for methyl ester). C₁₈1H₁₆F₃N₂O₄ requires 384. Ret. time 3.62 min.

### DESCRIPTION 22

### 3-[4-(Trifluoromethyl)phenyl]-1,4-diazaspiro[4.4]nonan-2-one

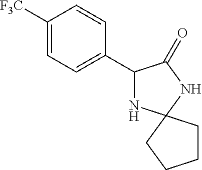

Cyclopentanone (3.32 mL, 37.6 mmol) was added to a stirred solution of 2-amino-2-[4-(trifluoromethyl)phenyl]acetamide D9 (8.2 g, 37.6 mmol) in methanol (80 mL). p-Toluenesulfonic acid monohydrate (71 mg, 0.38 mmol) was then added and the mixture left to stir under reflux for 18.5 h. The mixture was concentrated in vacuo and the solid residue partitioned between DCM and a dilute aqueous solution of sodium bicarbonate (1:4 saturated aqueous sodium bicarbonate solution: water). The layers were separated and the aqueous extracted twice with DCM. The DCM extracts were combined, dried (Na₂SO₄), filtered and concentrated in vacuo to give the title compound as a cream amorphous solid (9.89 g, 92%). ¹H NMR (d₆-DMSO) δ: 1.60-1.85 (7H, m), 2.25-2.90 (1H, m), 3.72 (1H, d), 4.66 (1H, d), 7.60-7.75 (4H, m), 8.60 (1H, br s). Mass Spectrum (Electrospray LC/MS). Found 285.2 (M+H)⁺. C₁₄H₁₅F₃N₂O requires 284.3. Ret. time 2.75 min.

### DESCRIPTION 23

### 3-[4-(Trifluoromethyl)phenyl]-1,4-diazaspiro[4.4]non-3-en-2-one

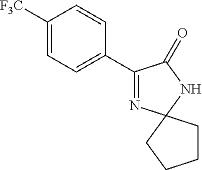

N-Bromosuccinimide (6.69 g, 37.6 mmol) was added to a stirred solution of 3-[4-(trifluoromethyl)phenyl]-1,4-diazaspiro[4.4]nonan-2-one D22 (9.89 g, 34.8 mmol) in DCM (320 mL). The mixture was left to stir at room temperature for 17.5 h. Saturated aqueous sodium bicarbonate solution (ca. 300 mL) was added and the mixture stirred at room temperature for 30 mins. The DCM layer was then separated and the aqueous extracted twice with DCM. The DCM extracts were combined, dried (MgSO₄), filtered and concentrated in vacuo to give a solid black residue. The residue was dissolved in DCM (300 mL) and saturated aqueous sodium bicarbonate solution (300 mL) was added. The mixture was then stirred vigorously for 18 h. The DCM layer was separated and the aqueous was extracted twice with DCM. The DCM extracts were combined, dried (MgSO₄), filtered and concentrated in vacuo to give a black residue. This residue was purified by silica gel chromatography eluting with 20-60% ethyl acetate/cyclohexane to give the title compound as cream amorphous solid (2.90 g, 30%). ¹H NMR (CDCl₃) δ: 1.90-2.20 (8H, m), 7.73 (2H, d), 8.53 (2H, d), 8.65 (1H, br s). Mass Spectrum (Electrospray LC/MS). Found 283.2 (M+H)⁺. C₁₄H₁₃F₃N₂O requires 282.3. Ret. time 3.20 min.

### DESCRIPTION 24

### Ethyl {2-oxo-3-[4-(trifluoromethyl)phenyl]-1,4-diazaspiro[4.4]non-3-en-1-yl}acetate

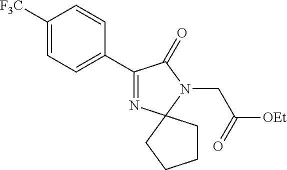

Ethyl bromoacetate (2.75 mL, 24.8 mmol) and then potassium carbonate (754 mg, 5.46 mmol) were added to a stirred solution of 3-[4-(trifluoromethyl)phenyl]-1,4-diazaspiro[4.4]non-3-en-2-one D23 (1.40 g, 4.96 mmol) in acetone (75 mL). The mixture was then stirred under reflux for 47 h. More potassium carbonate (754 mg, 5.46 mmol) was then added and the mixture left to stir for a further 20 h. The mixture was cooled to room temperature and partitioned between DCM and water. The DCM layer was separated and the aqueous layer extracted twice with DCM. The DCM extracts were combined, dried (MgSO₄), filtered and concentrated in vacuo to give a yellow oil (ca. 6.5 g). Purification by silica gel chromatography eluting with 20-60% ethyl acetate/cyclohexane gave the title compound as a white amorphous solid (1.46 g, 80%). ¹H NMR (CDCl₃) δ: 1.30 (3H, t), 1.82-2.05 (6H, m), 2.10-2.20 (2H, m), 4.19 (2H, s), 4.25 (2H, q), 7.72 (2H, d), 8.58 (2H, d). Mass Spectrum (Electrospray LC/MS). Found 369.1 (M+H)⁺. C₁₈H₁₉F₃N₂O₃ requires 368.4. Ret. time 3.56 min.

### DESCRIPTION 25

### {2-oxo-3-[4-(Trifluoromethyl)phenyl]-1,4-diazaspiro[4.4]non-3-en-1-yl}acetic acid

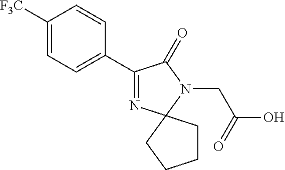

Sodium hydroxide (190 mg, 4.75 mmol) was added to a stirred solution of ethyl {2-oxo-3-[4-(trifluoromethyl)phenyl]-1,4-diazaspiro[4.4]non-3-en-1-yl}acetate D24 (1.46 g, 3.96 mmol) in water (45 mL)/methanol (14 mL). The mixture was then heated at 60° C. for 24 h. More sodium hydroxide (95 mg, 2.38 mmol) was added and the mixture stirred for a further 17 h. The mixture was cooled and then concentrated in vacuo. The residue was partitioned between ethyl acetate and dilute aqueous sodium bicarbonate solution (ca. 1:10 saturated aqueous sodium bicarbonate solution: water). The layers were separated and the ethyl acetate layer was extracted twice with dilute aqueous sodium bicarbonate solution. The aqueous extracts were combined and acidified to pH 2 with 2M HCl_{(aq)}. The aqueous was then extracted 3 times with DCM. The DCM extracts were combined, dried (MgSO₄), filtered and concentrated in vacuo to give a white amorphous solid (1.15 g, 85%). ¹H NMR (CDCl₃) δ: 1.80-2.05 (6H, m), 2.10-2.20 (2H, m), 4.25 (2H, s), 7.72 (2H, d), 8.54 (2H, d). Mass Spectrum (Electrospray LC/MS). Found 339.1 (M−H)⁻. C₁₆H₁₅F₃N₂O₃ requires 340.3. Ret. time 3.37 min.

### EXAMPLE 1

### N-(1-Hydroxy-cyclohexylmethyl)-2-[2-oxo-3-(4-trifluoromethyl-phenyl)-1,4-diaza-spiro[4.4]non-3-en-1-yl]-acetamide

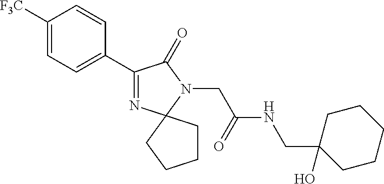

Diisopropylethylamine (16.5 μL, 0.095 mmol) and 1-aminomethylcyclohexanol hydrochloride (13 mg, 0.104 mmol) were added to a stirred solution of {2-oxo-3-[4-(trifluoromethyl)phenyl]-1,4-diazaspiro[4.4]non-3-en-1-yl}acetic acid D25 (32 mg, 0.095 mmol) in DCM (1 mL). HATU was then added as a solution in DMF (800 mg/mL, 50 μL, 0.104 mmol). The mixture was then left to stir at room temperature for 46 h. More DCM (1 mL) was added and then more diisopropylethylamine (16.5 μL, 0.095 mmol), 1-aminomethylcyclohexanol hydrochloride (13 mg, 0.104 mmol) and HATU (as a solid, 40 mg, 0.104 mmol) were added and the mixture left to stir for a further 2.5 h. The mixture was concentrated by flow of N₂ gas and then purified by mass directed auto-purification chromatography to give the title compound (13 mg, 31%). ¹H NMR (d₆-DMSO) δ: 1.12-1.58 (10H, m), 1.58-1.70 (2H, m), 1.81-2.11 (6H, m), 3.03 (2H, d), 4.15 (2H, s), 4.28 (1H, s), 7.78-7.98 (3H, m), 8.48 (2H, d). Mass Spectrum (Electrospray LC/MS). Found 452.0 (M+H)⁺. C₂₃H₂₈F₃N₃O₃ requires 451.5. Ret. time 3.25 min.

### EXAMPLE 2

### N-(2-Chloro-benzyl)-2-[2-oxo-3-(4-trifluoromethyl-phenyl)-1,4-diaza-spiro[4.4]non-3-en-1-yl]-acetamide

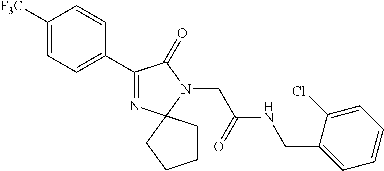

Diisopropylethylamine (12.7 μL, 0.073 mmol) and 2-chlorobenzylamine (9.7 μL, 0.080 mmol) were added to a stirred solution of {2-oxo-3-[4-(trifluoromethyl)phenyl]-1,4-diazaspiro[4.4]non-3-en-1-yl}acetic acid D25 (25 mg, 0.073 mmol) in DCM (1 mL). HATU (31 mg, 0.080 mmol) was then added and the mixture was left to stir at room temperature for 62.5 h. The mixture was concentrated by flow of N₂ gas and then purified by UV-directed preparative HPLC purification chromatography to give the title compound (8.2 mg, 24%). ¹H NMR (CDCl₃) δ: 1.68-1.82 (2H, m), 1.89-2.22 (6H, m), 4.13 (2H, s), 4.51 (2H, d), 6.97 (1H, br s), 7.18-7.24 (2H, m), 7.30-7.38 (2H, m), 7.73 (2H, d), 8.53 (2H, d). Mass Spectrum (Electrospray LC/MS). Found 464.2 (M+H)⁺. C₂₃H₂₁ClF₃N₃O₂ requires 463.88. Ret. time 3.63 min.

### EXAMPLE 3

### 2-{2-oxo-3-[4-(trifluoromethyl)phenyl]-1,4-diazaspiro[4.5]dec-3-en-1-yl}-N-(2-thienylmethyl)acetamide

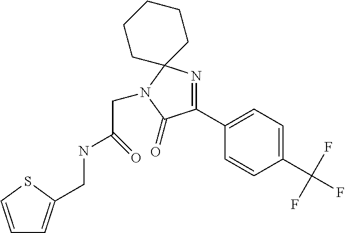

Polymer supported EDC (60 mg, loading 1.38 mmol/g, supplier: Polymer Laboratories) was sonicated for 30 min in a solution of HOAT in DCM:THF 1:1 (750 uL of a stock solution containing 570 mg HOAT in 330 ml DCM:THF 1:1). To this mixture {2-oxo-3-[4-(trifluoromethyl)phenyl]-1,4-diazaspiro[4.5]dec-3-en-1-yl}acetic acid D13 (17.7 mg, 0.05 mmol) in NMP:THF 1:3 (2504) and 2-aminomethylthiophene (5.7 mg, 0.06 mmol) in DCM (2504) were added. The mixture was shaken over the weekend, following which polymer supported isocyanate (68 mg, loading 1.11 mmol/g, supplier: Argonaut Technologies) and polymer supported carbonate (68 mg, loading 1.11 mmol/g, supplier: Argonaut Technologies) were added and the mixture shaken for a further 24 h. The mixture was filtered and solvent removed using a Genevac (model HT-12) and purified by mass directed autopurification to give the title compound (11.9 mg, 50%). Mass Spectrum (Electrospray LC/MS). Found 450 (M+H)⁺. C₂₂H₂₂F₃N₃O₂S requires 449. Ret. time 3.5 min.

The compounds in the table below were prepared using similar methods to that described in Example 3

| | | Mass spectrum | |
|---|---|---|---|
| | | (Electrospray | |
| | | LC/MS), API⁺ | |
| Ex | Structure | Ret. time (min) | Name |
| 4 | 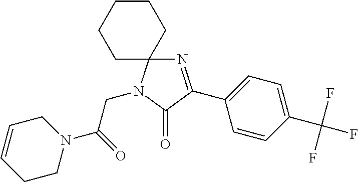 | Found 420 (MH⁺) C₂₂H₂₄F₃N₃O₂ requires 419; 3.52 | 1-[2-(3,6-dihydro-1(2H)- pyridinyl)-2-oxoethyl]-3- [4- (trifluoromethyl)phenyl]- 1,4-diazaspiro[4.5]dec- 3-en-2-one |
| 5 | 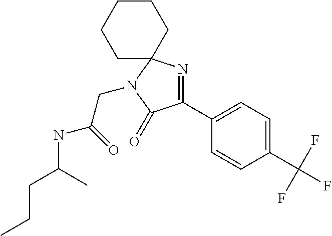 | Found 424 (MH⁺) C₂₂H₂₈F₃N₃O₂ requires 423; 3.62 | N-(1-methylbutyl)-2-{2- oxo-3-[4- (trifluoromethyl)phenyl]- 1,4-diazaspiro[4.5]dec- 3-en-1-yl}acetamide |
| 6 | 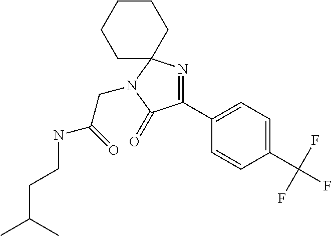 | Found 424 (MH⁺) C₂₂H₂₈F₃N₃O₂ requires 423; 3.62 | N-(3-methylbutyl)-2-{2- oxo-3-[4- (trifluoromethyl)phenyl]- 1,4-diazaspiro[4.5]dec- 3-en-1-yl}acetamide |
| 7 | 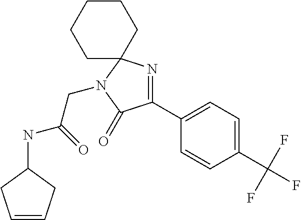 | Found 420 (MH⁺) C₂₂H₂₄F₃N₃O₂ requires 419; 3.89 | N-3-cyclopenten-1-yl-2- {2-oxo-3-[4- (trifluoromethyl)phenyl]- 1,4-diazaspiro[4.5]dec- 3-en-1-yl}acetamide |
| 8 | 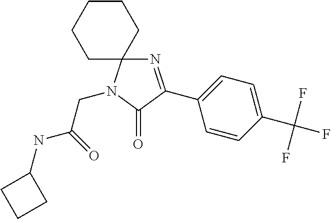 | Found 408 (MH⁺) C₂₁H₂₄F₃N₃O₂ requires 407; 3.86 | N-cyclobutyl-2-{2-oxo-3- [4- (trifluoromethyl)phenyl]- 1,4-diazaspiro[4.5]dec- 3-en-1-yl}acetamide |
| 9 | 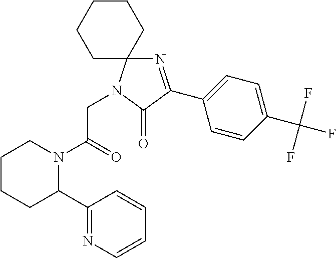 | Found 499 (MH⁺) C₂₇H₂₃F₃N₄O₂ requires 498; 3.48 | 1-{2-oxo-2-[2-(2- pyridinyl)-1- piperidinyl]ethyl}-3-[4- (trifluoromethyl)phenyl]- 1,4-diazaspiro[4.5]dec- 3-en-2-one |
| 10 | 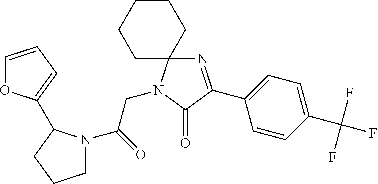 | Found 474 (MH⁺) C₂₅H₂₆F₃N₃O₃ requries 473; 3.65 | 1-{2-[2-(2-furanyl)-1- pyrrolidinyl]-2-oxoethyl}- 3-[4- (trifluoromethyl)phenyl]- 1,4-diazaspiro[4.5]dec- 3-en-2-one |
| 11 | 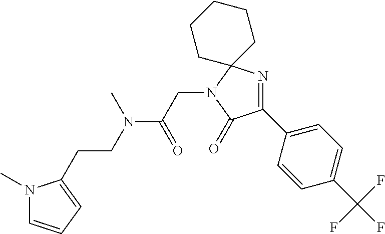 | Found 475 (MH⁺) C₂₅H₂₃F₃N₄O₂ requires 474; 3.6 | N-methyl-N-[2-(1- methyl-1H-pyrrol-2- yl)ethyl]-2-{2-oxo-3-[4- (trifluoromethyl)phenyl]- 1,4-diazaspiro[4.5]dec- 3-en-1-yl}acetamide |
| 12 | 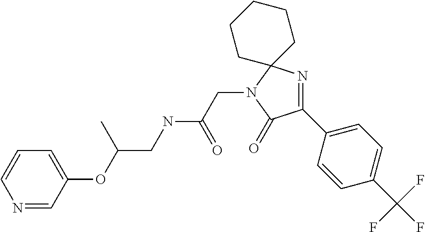 | Found 489 (MH⁺) C₂₅H₂₇F₃N₄O₃ requires 488; 2.92 | 2-{2-oxo-3-[4- (trifluoromethyl)phenyl]- 1,4-diazaspiro[4.5]dec- 3-en-1-yl}-N-[2-(3- pyridinyloxy)propyl] acetamide |
| 13 | 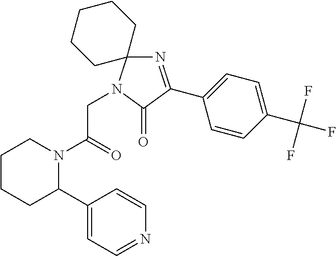 | Found 499 (MH⁺) C₂₇H₂₉F₃N₄O₂ requires 498; 2.92 | 1-{2-oxo-2-[2-(4- pyridinyl)-1- piperidinyl]ethyl}-3-[4- (trifluoromethyl)phenyl]- 1,4-diazaspiro[4.5]dec- 3-en-2-one |
| 14 | 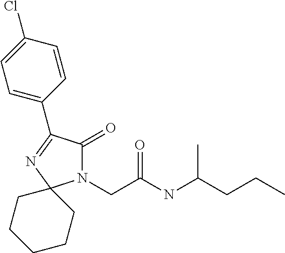 | Found 390 (MH⁺) C₂₁H₂₈³⁵ClN₃O₂ requries 389; 3.61 | 2-[3-(4-chlorophenyl)-2- oxo-1,4- diazaspiro[4.5]dec-3-en- 1-yl]-N-(1- methylbutyl)acetamide |
| 15 | 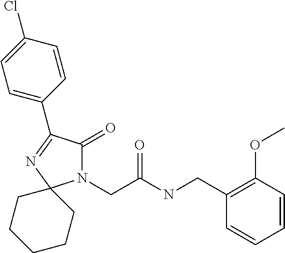 | Found 440 (MH⁺) C₂₄H₂₆³⁵ClN₃O₃ requires 439 | 2-[3-(4-chlorophenyl)-2- oxo-1,4- diazaspiro[4.5]dec-3-en- 1-yl]-N-{[2- (methyloxy)phenyl] methyl}acetamide |
| 16 | 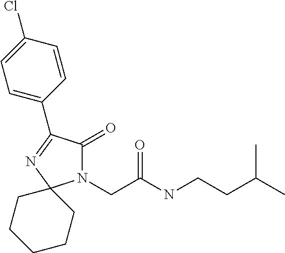 | Found 390 (MH⁺) C₂₁H₂₈³⁵ClN₃O₂ requries 389; 3.63 | 2-[3-(4-chlorophenyl)-2- oxo-1,4- diazaspiro[4.5]dec-3-en- 1-yl]-N-(3- methylbutyl)acetamide |
| 17 | 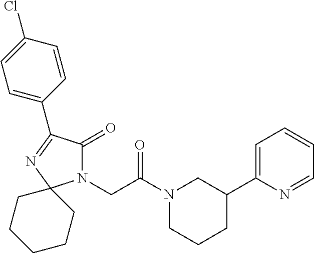 | Found 465 (MH⁺) C₂₆H₂₉³⁵ClN₄O₂ requires 464; 2.95 | 3-(4-chlorophenyl)-1-{2- oxo-2-[3-(2-pyridinyl)-1- piperidinyl]ethyl}-1,4- diazaspiro[4.5]dec-3-en- 2-one |
| 18 | 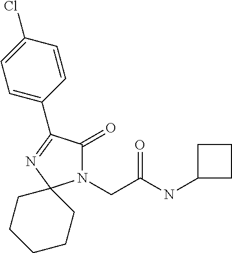 | Found 374 (MH⁺) C₂₀H₂₄³⁵ClN₃O₂ requires 373; 3.45 | 2-[3-(4-chlorophenyl)-2- oxo-1,4- diazaspiro[4.5]dec-3-en- 1-yl]-N- cyclobutylacetamide |
| 19 | 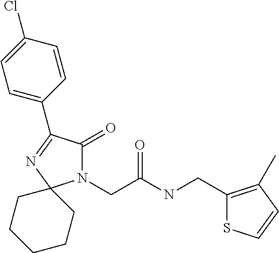 | Found 430 (MH⁺) C₂₂H₂₄³⁵ClN₃O₂S requires 429; 3.6 | 2-[3-(4-chlorophenyl)-2- oxo-1,4- diazaspiro[4.5]dec-3-en- 1-yl]-N-[(3-methyl-2- thienyl)methyl]acetamide |
| 20 | 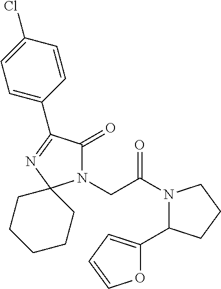 | Found 440 (MH⁺) C₂₄H₂₆³⁵ClN₃O₃ requires 439; 3.64 | 3-(4-chlorophenyl)-1-{2- [2-(2-furanyl)-1- pyrrolidinyl]-2-oxoethyl}- 1,4-diazaspiro[4.5]dec- 3-en-2-one |
| 21 | 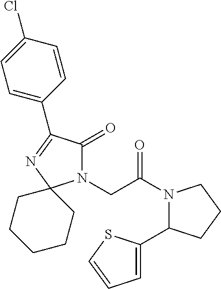 | Found 456 (MH⁺) C₂₄H₂₆³⁵ClN₃O₂S requires 455; 3.76 | 3-(4-chlorophenyl)-1-{2- oxo-2-[2-(2-thienyl)-1- pyrrolidinyl]ethyl}-1,4- diazaspiro[4.5]dec-3-en- 2-one |
| 22 | 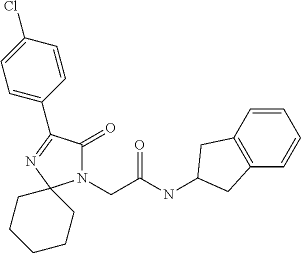 | Found 435 (MH⁺) C₂₅H₂₆³⁵ClN₃O₂ requires 435; 3.65 | 2-[3-(4-chlorophenyl)-2- oxo-1,4- diazaspiro[4.5]dec-3-en- 1-yl]-N-(2,3-dihydro-1H- inden-2-yl)acetamide |
| 23 | 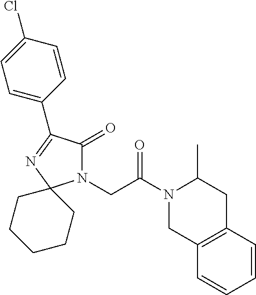 | Found 450 (MH⁺) C₂₆H₂₈³⁵ClN₃O₂ requires 449; 3.8 | 3-(4-chlorophenyl)-1-[2- (3-methyl-3,4-dihydro- 2(1H)-isoquinolinyl)-2- oxoethyl]-1,4- diazaspiro[4.5]dec-3-en- 2-one |

### EXAMPLE 24

### 1-{2-oxo-2-[3-(2-pyridinyl)-1-piperidinyl]ethyl}-3-{4-[(trifluoromethyl)oxy]phenyl}-1,4-diazaspiro[4.5]dec-3-en-2-one

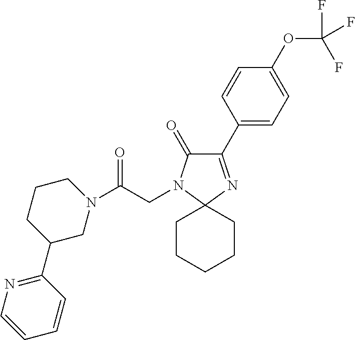

A solution of (2-oxo-3-{4-[(trifluoromethyl)oxy]phenyl}-1,4-diazaspiro[4.5]dec-3-en-1-yl)acetyl chloride (prepared from (2-oxo-3-{4-[(trifluoromethyl)oxy]phenyl}-1,4-diazaspiro[4.5]dec-3-en-1-yl)acetic acid (0.104 g 0.282 mmol) according to the method of D21) in DCM was added to a solution of 2-(3-piperidinyl)pyridine (0.055 g 0.34 mmol) and triethylamine (0.078 ml) in DCM (total volume used 10 ml). The mixture was stirred for 16 h under argon, diluted with DCM, washed with saturated sodium hydrogen carbonate solution, water and brine, dried (MgSO₄) and evaporated. The crude product was chromatographed (silica gel; 0-100% pentane/ethyl acetate) to give the title compound as a white foam (0.100 g). ¹H NMR (CDCl₃) evidence for rotamers δ: 1.24-2.18 (14H, m) 2.84-2.99 (2H, m), 3.15 and 3.53 (1H, t), 3.95-4.45 (m) and 4.70 (total 4H, d), 7.12-7.30 ((4H, m), 7.59-7.66 (1H, m and 8.52-8.56 (3H, m). Mass Spectrum (Electrospray LC/MS). Found 515 (MH⁺). C₂₇H₂₉F₃N₄O₃ requires 514. Ret. time 2.97 min.

The compounds in the table below were prepared using similar methods to that described in Example 24:

| | | Mass spectrum | |
|---|---|---|---|
| | | (Electrospray | |
| | | LC/MS), API⁺ | |
| Ex | Structure | Ret. time (min) | Name |
| 25 | 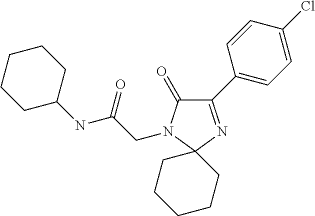 | Found 402 (MH⁺) C₂₂H₂₈³⁵ClN₃O₂ requires 401; 3.61 | 2-[3-(4-chlorophenyl)-2-oxo- 1,4-diazaspiro[4.5]dec-3-en- 1-yl]-N-cyclohexylacetamide |
| 26 | 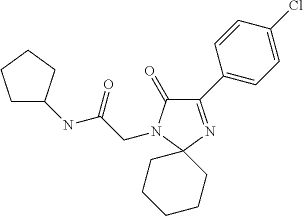 | Found 388 (MH⁺) C₂₁H₂₆³⁵ClN₃O₂ requires 387; 3.40 | 2-[3-(4-chlorophenyl)-2-oxo- 1,4-diazaspiro[4.5]dec-3-en- 1-yl]-N-cyclopentylacetamide |
| 27 | 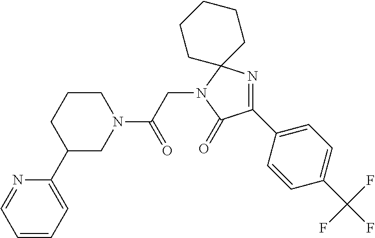 | Found 499 (MH⁺) C₂₇H₂₉F₃N₄O₂ requires 498; 2.92 | 1-{2-oxo-2-[3-(2-pyridinyl)-1- piperidinyl]ethyl}-3-[4- (trifluoromethyl)phenyl]-1,4- diazaspiro[4.5]dec-3-en-2- one |
| 28 | 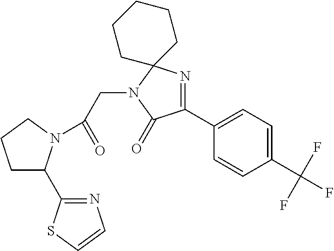 | Found 491 (MH⁺) C₂₄H₂₅F₃N₄O₂S requires 490, 3.33 | 1-{2-oxo-2-[2-(1,3-thiazol-2- yl)-1-pyrrolidinyl]ethyl}-3-[4-[4 (trifluoromethyl)phenyl]-1,4- diazaspiro[4.5]dec-3-en-2- one |
| 29 | 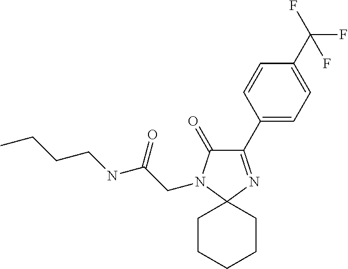 | Found 410 (MH⁺) C₂₁H₂₆F₃N₃O₂ requires 409; 3.48 | N-butyl-2-{2-oxo-3[4- (trifluoromethyl)phenyl]-1,4- diazaspiro[4.5]dec-3-en-1- yl}acetamide |
| 30 | 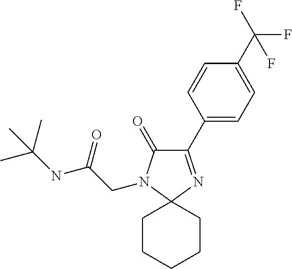 | Found 410 (MH⁺) C₂₁H₂₆F₃N₃O₂ requires 409; 3.56 | N-(1,1-dimethylethyl)-2-{2- oxo-3-[4- (trifluoromethyl)phenyl]-1,4- diazaspiro[4.5]dec-3-en-1- yl}acetamide |
| 31 | 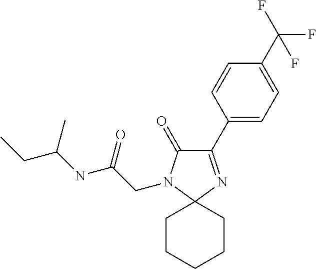 | Found 410 (MH⁺) C₂₁H₂₆F₃N₃O₂ requires 409; 3.44 | N-(1-methylpropyl)-2-{2-oxo- 3-[4-(trifluoromethyl)phenyl]- 1,4-diazaspiro[4.5]dec-3-en- 1-yl}acetamide |
| 32 | 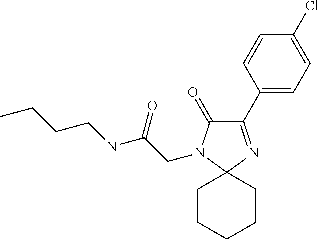 | Found 376 (MH⁺) C₂₀H₂₆³⁵ClN₃O₂ requires 375; 3.40 | N-butyl-2-[3-(4-chlorophenyl)- 2-oxo-1,4-diazaspiro[4.5]dec- 3-en-1-yl]acetamide |
| 33 | 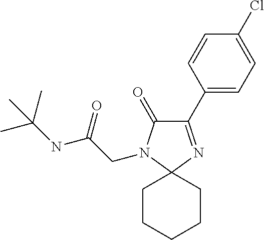 | Found 376 (MH⁺) C₂₀H₂₆³⁵ClN₃O₂ requires 375; 3.48 | 2-[3-(4-chlorophenyl)-2-oxo- 1,4-diazaspiro[4.5]dec-3-en- 1-yl]-N-(1,1- dimethylethyl)acetamide |
| 34 | 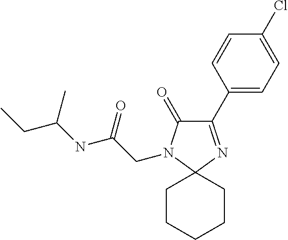 | Found 376 (MH⁺) C₂₀H₂₆³⁵ClN₃O₂ requires 375; 3.36 | 2-[3-(4-chlorophenyl)-2-oxo- 1,4-diazaspiro[4.5]dec-3-en- 1-yl]-N-(1- methylpropyl)acetamide |
| 35 | 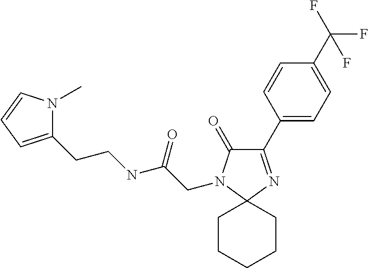 | Found 461 (MH⁺) C₂₄H₂₇F₃N₄O₂ requires 460; 3.41 | N-[2-(1-methyl-1H-pyrrol-2- yl)ethyl]-2-{2-oxo-3-[4- (trifluoromethyl)phenyl]-1,4- diazaspiro[4.5]dec-3-en-1- yl}acetamide |
| 36 | 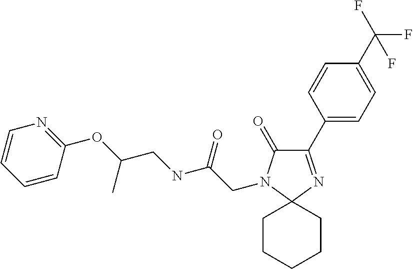 | Found 489 (MH⁺) C₂₅H₂₇F₃N₄O₃ requires 488; 2.65 | 2-{2-oxo-3-[4- (trifluoromethyl)phenyl]-1,4- diazaspiro[4.5]dec-3-en-1-yl}- N-[2-(2- pyridinyloxy)propyl]acetamide |
| 37 | 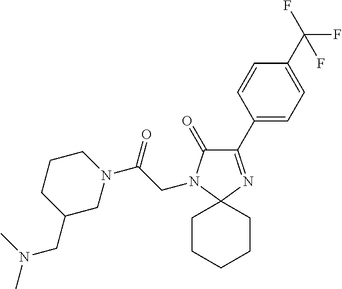 | Found 479 (MH⁺) C₂₅H₃₃F₃N₄O₂ requires 478; 2.26 | 1-(2-{3- [(dimethylamino)methyl]-1- piperidinyl}-2-oxoethyl)-3-[4- (trifluoromethyl)phenyl]-1,4- diazaspiro[4.5]dec-3-en-2- one |
| 38 | 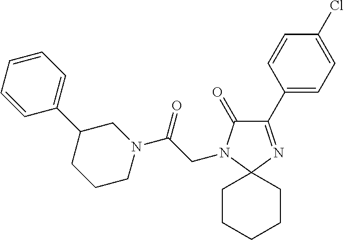 | Found 464 (MH⁺) C₂₇H₃₀ClN₃O₂ requires 464; 3.76 | 3-(4-chlorophenyl)-1-[2-oxo- 2-(3-phenyl-1- piperidinyl)ethyl]-1,4- diazaspiro[4.5]dec-3-en-2- one |
| 39 | 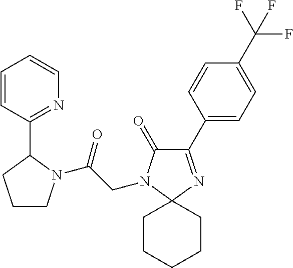 | Found 485 (MH⁺) C₂₆H₂₇F₃N₄O₂ requires 484; 2.99 | 1-{2-oxo-2-[2-(2-pyridinyl)-1- pyrrolidinyl]ethyl}-3-[4- (trifluoromethyl)phenyl]-1,4- diazaspiro[4.5]dec-3-en-2- one |
| 40 | 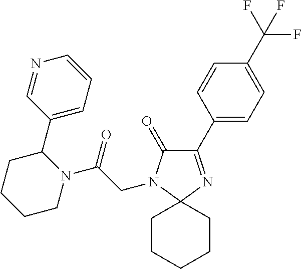 | Found 499 (MH⁺) C₂₇H₂₉F₃N₄O₂ rerquries 498; 2.83 | 1-{2-oxo-2-[2-(3-pyridinyl)-1- piperidinyl]ethyl}-3-[4- (trifluoromethyl)phenyl]-1,4- diazaspiro[4.5]clec-3-en-2- one |
| 41 | 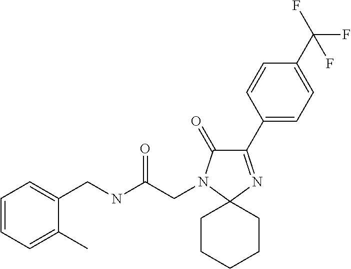 | Found 458 (MH⁺) C₂₅H₂₆F₃N₃O₂ requires 457; 3.57 | N-[(2-methylphenyl)methyl]- 2-{2-oxo-3-4- (trifluoromethyl)phenyl]-1,4- diazaspiro[4.5]dec-3-en-1- yl}acetamide |
| 42 | 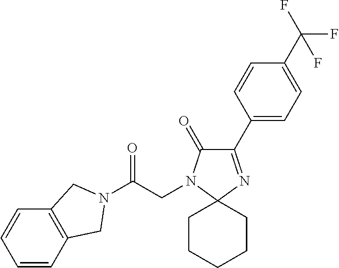 | Found 456 (MH⁺) C₂₅H₂₆F₃N₃O₂ requires 455; 3.54 | 1-[2-(1,3-dihydro-2H-isoindol- 2-yl)-2-oxoethyl]-3-[4- (trifluoromethyl)phenyl]-1,4- diazaspiro[4.5]dec-3-en-2- one |

### EXAMPLE 43

### 2-[3-(4-chlorophenyl)-2-oxo-1,4-diazaspiro[4.5]dec-3-en-1-yl]-N-[2-methylphenyl)methyl]acetamide

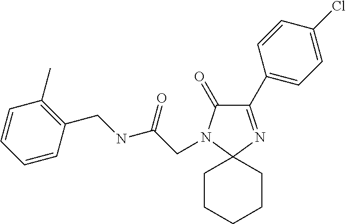

2-Methylbenzylamine (0.045 g, 0.37 mmol), [3-(4-Chlorophenyl)-2-oxo-1,4-diazaspiro[4.5]dec-3-en-1-yl]acetic acid D6 (0.120 g, 0.37 mmol), EDC (0.143 g, 0.75 mmol) and 1-hydroxybenzotriazole (0.05 g, 0.37 mmol) were combined in DCM (5 ml) and stirred overnight. Saturated sodium hydrogen carbonate was added and the mixture extracted twice with DCM. The organic extracts were combined, dried (MgSO₄) and evaporated to dryness. The crude residue was column chromatographed (silica gel eluant pentane →30% ethyl acetate/pentane) to give the title compound (0.13 g). ¹H NMR (CDCl₃) δ: 1.24-1.30 (4H, m) 1.80-2.05 (6H, m), 2.28 (3H, s), 4.13 (2H, s), 4.41 (2H, d), 7.16 (4H, m), 7.45 (2H, d) and 8.40 (2H, d). Mass Spectrum (Electrospray LC/MS). Found 424 (MH⁺). C₂₄H₂₆³⁵ClN₃O₂ requires 423. Ret. time 3.57 min.

The compounds in the table below were prepared using similar methods to that described in example 43:

| | | Mass | |
|---|---|---|---|
| | | spectrum | |
| | | (Electrospray | |
| | | LC/MS), API⁺ | |
| | | Ret. time | |
| Ex | Structure | (min) | Name |
| 44 | 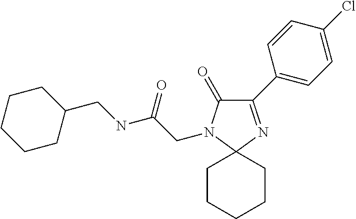 | Found 416 (MH⁺) C₂₃H₃₀³⁵ClN₃O₂ requires 415; 3.74 | 2-[3-(4-chlorophenyl)-2-oxo- 1,4-diazaspiro[4.5]dec-3-en- 1-yl]-N- (cyclohexylmethyl)acetamide |
| 45 | 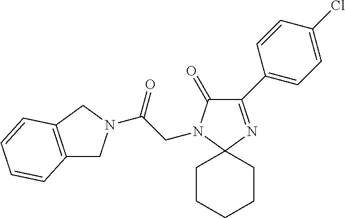 | Found 422 (MH⁺) C₂₄H₂₄³⁵ClN₃O₂ requires 421; 3.50 | 3-(4-chlorophenyl)-1-[2-(1,3- dihydro-2H-isoindol-2-yl)-2- oxoethyl]-1,4- diazaspiro[4.5]dec-3-en-2- one |

1.-21. (canceled)

22. A compound of formula (I) or a salt or solvate thereof: 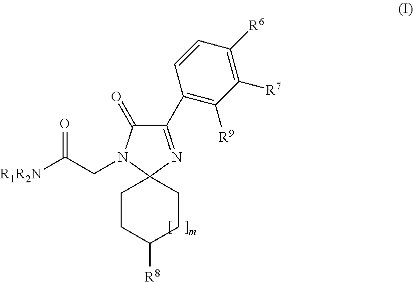
wherein:
R⁶ is haloC₁-C₄alkoxy, haloC₁-C₄alkyl, or chloro;
R⁷ is H, C₁-C₄alkyl, C₁-C₄alkoxy, haloC₁-C₄alkyl, haloC₁-C₄alkoxy, halo, cyano, C₁-C₄alkoxyC₁-C₄alkyl or C₁-C₄alkoxyC₁-C₄alkoxyl;
R⁸ is hydrogen or methyl;
R⁹ is H or F;
m is 0, 1 or 2;
and wherein when R⁶ is haloC₁-C₄alkoxy or haloC₁-C₄alkyl and m is 0, then R¹ is hydrogen and R² is a group —(C₁-C₆alkyl)-Y wherein Y is a 6-membered carbocyclic saturated or unsaturated ring, optionally substituted by a halogen or a hydroxyl;
and wherein when R⁶ is haloC₁-C₄alkoxy or haloC₁-C₄alkyl and m is 1 or 2, then:
R¹ and R² are independently selected from the group consisting of:
hydrogen;
C₁-C₆alkyl;
C₃-C₆cycloalkyl;
C₁-C₆alkylC₄-C₆cycloalkyl (wherein the cycloalkyl is optionally substituted by —OH);
C₃-C₆cycloalkyl fused with phenyl or fused with an unsaturated C₃-C₆-carbocyclic group;
CH₂—CHMe-O-pyridyl; and
—(CH₂)ₙ—Z wherein n is 1, 2, 3, 4, 5 or 6 or Z is pyrrolyl optionally substituted by C₁-C₆alkyl, phenyl substituted by halo, C₁-C₆alkyl or C₁-C₆alkoxy, or thienyl optionally substituted by a C₁-C₆alkyl group;
with the proviso R¹ and R² are not both H;
or R¹ and R² together with the nitrogen atom to which they are attached form:
an azetidinyl, pyrrolidinyl, piperidyl or azepanyl group, each of which is optionally substituted by C₁-C₆alkyl, and each of which is also optionally fused with a 5- or 6 membered aromatic or heteroaromatic group containing one or two heteratoms selected from O, S or N; or
pyrrolidinyl which is substituted at the 2 position with 2-pyridyl or a 5 membered heteroaromatic group containing one or two heteroatoms selected from O, S or N, or CH₂N(CH₃)₂; or
piperidyl which is substituted at the 2- or 3-position by a 5- or 6-membered aromatic or heteroaromatic group;
and wherein, when R⁶ is chloro, m is 0, 1, or 2, then:
R¹ and R² are independently selected from the group consisting of:
hydrogen;
C₁-C₆alkyl;
C₃-C₆cycloalkyl;
C₄-C₆cycloalkylC₁-C₆alkyl;
C₃-C₆cycloalkyl fused with phenyl or fused with an unsaturated C₃-C₆-carbocyclic group;
a group —CH₂—CHMe-O-pyridyl; and
a group —(CH₂)n-Z wherein n is 1, 2, 3, 4, 5 or 6 and Z is phenyl substituted by a C₁-C₆alkyl or a C₁-C₆alkoxy group, or Z is thienyl substituted by a C₁-C₆alkyl group;
with the proviso R¹ and R² are not both H;
or R¹ and R² together with the nitrogen atom to which they are attached form:
an azetidinyl, pyrrolidinyl, piperidyl or azepanyl group, each of which being optionally substituted by C₁-C₆alkyl, and each of which is also optionally fused with phenyl;
a pyrrolidinyl which is substituted at the 2 position with a 5 membered heteroaromatic group containing one heteroatom which is O, S or N; or
piperidyl which is substituted at the 2- or 3-position by a 5- or 6-membered aromatic or heteroaromatic group containing one or two heteratoms selected from O, S and N.

23. A compound as claimed in claim 22 wherein R⁶ is trifluoromethoxy, trifluoromethyl or chloro.

24. A compound as claimed in claim 22 wherein R⁷ is hydrogen.

25. A compound as claimed in claim 22 wherein m is 0 or 1.

26. A compound as claimed in claim 22 wherein R⁸ is hydrogen.

27. A compound as claimed in claim 22 wherein, when R⁶ is haloC₁-C₄alkoxy or haloC₁-C₄alkyl and m is 0, then R¹ is hydrogen and R² is a group —CH₂—Y wherein Y is a 6-membered carbocyclic saturated or unsaturated ring, substituted by a halogen or a hydroxyl group.

28. A compound as claimed in claim 22 wherein R¹ is hydrogen and R² is a group —CH₂—Y wherein Y is 1-hydroxy-cyclohexyl or 2-chlorophenyl.

29. A compound as claimed in claim 22 wherein, when R⁶ is haloC₁-C₄alkoxy or haloC₁-C₄alkyl and m is 1 or 2, then:
R₁ and R₂ are independently selected from the group consisting of:
Hydrogen;
C₁-C₅alkyl;
C₄-C₅cycloalkyl;
—CH₂CHMeO-pyridinyl;
—CH₂-thienyl;
2-methylphenylmethyl; and
—CH₂CH₂-pyrrolyl (optionally N-substituted by methyl);
provided that R₁ and R₂ are not both H;
or R₁ and R₂, together with the nitrogen to which they are attached form:
4,5-dehydropiperidinyl;
piperidinyl substituted by pyridinyl or dimethylaminomethyl;
pyrrolidinyl substituted by furyl, thiazolyl, or pyridyl; or
isoindolinyl.

30. A compound as claimed in claim 22 wherein:
R₁ and R₂ are independently selected from the group consisting of:
hydrogen, methyl, n-butyl, t-butyl, 1-methylpropyl, 1-methylbutyl, 3-methylbutyl, cyclobutyl, 3,4-dehydrocyclopentyl, —CH₂CHMeO-pyridin-3-yl, —CH₂CHMeO-pyridin-2-yl, —CH₂-thien-2-yl, 2-methylphenylmethyl, —CH₂CH₂-pyrrol-2-yl, and —CH₂CH₂-1-methyl-pyrrol-2-yl;
provided that R₁ and R₂ are not both H; or
R₁ and R₂, together with the nitrogen to which they are attached, form:
4,5-dehydropiperidinyl;
piperidinyl substituted at the 2-position by pyridin-2-yl, pyridin-3-yl or pyridin-4-yl, or substituted at the 3-position by pyridin-2-yl or dimethylaminomethyl; or
pyrrolidinyl substituted at the 2-position furan-2-yl, thiazol-2-yl, or pyridin-2-yl; or
isoindolinyl.

31. A compound as claimed in claim 22 wherein, when R⁶ is chloro, m is 0, 1, or 2, then:
R¹ and R² are independently selected from the group consisting of:
hydrogen, C₄-C₅alkyl, C₄-C₆cycloalkyl, C₁-C₆alkylC₄-C₆cycloalkyl, C₃-C₆cycloalkyl fused with phenyl, and
a group —CH₂—Z wherein Z is phenyl substituted by a C₁-C₆alkyl or C₁-C₆ alkoxy group, or Z is a thienyl substituted by a C₁-C₆alkyl group;
with the proviso R¹ and R² are not both H;
or R¹ and R², together with the nitrogen atom to which they are attached, form C₁-C₆alkyl-substituted dihydroisoquinolinyl, or dihydroisoindolyl;
or R¹ and R², together with the nitrogen atom to which they are attached, form:
a pyrrolidinyl which is substituted at the 2-position with furyl or thienyl, or
piperidyl which is substituted at the 3-position by pyridine or phenyl; and
R⁷, R⁸ and R⁹ are as defined in claim 1.

32. A compound as claimed in claim 31 wherein:
R¹ and R² are independently selected from the group consisting of:
hydrogen, 1-methylbutyl, 3-methylbutyl, n-butyl, t-butyl, 1-methylpropyl, cyclobutyl, cyclopentyl, cyclohexyl, —CH₂-cyclohexyl, 2,3-dihydro-1H-inden-2-yl, and
a group —CH₂—Z wherein Z is 2-methylphenyl, 2-methoxyphenyl, or 3-methyl-2-thienyl;
with the proviso R¹ and R² are not both H;
or R¹ and R², together with the nitrogen atom to which they are attached, form 3-methyl-3,4-dihydro-2(1H)-isoquinolin-2-yl, or 1,3-dihydro-2H-isoindol-2-yl;
or R¹ and R², together with the nitrogen atom to which they are attached, form:
a pyrrolidinyl which is substituted at the 2 position with 2-furyl or 2-thienyl, or
a piperidyl which is substituted at the 3-position by 2-pyridine or phenyl; and
R⁷, R⁸ and R⁹ are as defined in claim 1.

33. A compound as claimed in claim 22, which is the group consisting of:
N-(1-Hydroxy-cyclohexylmethyl)-2-[2-oxo-3-(4-trifluoromethyl-phenyl)-1,4-diaza-spiro[4.4]non-3-en-1-yl]-acetamide;
N-(2-Chloro-benzyl)-2-[2-oxo-3-(4-trifluoromethyl-phenyl)-1,4-diaza-spiro[4.4]non-3-en-1-yl]-acetamide;
2-{2-oxo-3-[4-(trifluoromethyl)phenyl]-1,4-diazaspiro[4.5]dec-3-en-1-yl}N-(2-thienylmethyl)acetamide;
1-[2-(3,6-dihydro-1(2H)-pyridinyl)-2-oxoethyl]-3-[4-(trifluoromethyl)phenyl]-1,4-diazaspiro[4.5]dec-3-en-2-one;
N-(1-methylbutyl)-2-{2-oxo-3-[4-(trifluoromethyl)phenyl]-1,4-diazaspiro[4.5]dec-3-en-1-yl}acetamide;
N-(3-methylbutyl)-2-{2-oxo-3-[4-(trifluoromethyl)phenyl]-1,4-diazaspiro[4.5]dec-3-en-1-yl}acetamide;
N-3-cyclopenten-1-yl-2-{2-oxo-3-[4-(trifluoromethyl)phenyl]-1,4-diazaspiro[4.5]dec-3-en-1-yl}acetamide;
N-cyclobutyl-2-{2-oxo-3-[4-(trifluoromethyl)phenyl]-1,4-diazaspiro[4.5]dec-3-en-1-yl}acetamide;
1-{2-oxo-2-[2-(2-pyridinyl)-1-piperidinyl]ethyl}-3-[4-(trifluoromethyl)phenyl]-1,4-diazaspiro[4.5]dec-3-en-2-one;
1-{2-[2-(2-furanyl)-1-pyrrolidinyl]-2-oxo ethyl}-3-[4-(trifluoromethyl)phenyl]-1,4-diazaspiro[4.5]dec-3-en-2-one;
N-methyl-N-[2-(1-methyl-1H-pyrrol-2-yl)ethyl]-2-{2-oxo-3-[4-(trifluoromethyl)phenyl]-1,4-diazaspiro[4.5]dec-3-en-1-yl}acetamide;
2-{2-oxo-3-[4-(trifluoromethyl)phenyl]-1,4-diazaspiro[4.5]dec-3-en-1-yl}N-[2-(3-pyridinyloxy)propyl]acetamide;
1-{2-oxo-2-[2-(4-pyridinyl)-1-piperidinyl]ethyl}-3-[4-(trifluoromethyl)phenyl]-1,4-diazaspiro[4.5]dec-3-en-2-one;
2-[3-(4-chlorophenyl)-2-oxo-1,4-diazaspiro[4.5]dec-3-en-1-yl]-N-(1-methylbutyl)acetamide;
2-[3-(4-chlorophenyl)-2-oxo-1,4-diazaspiro[4.5]dec-3-en-1-yl]-N-{[2-(methyloxy)phenyl]methyl}acetamide;
2-[3-(4-chlorophenyl)-2-oxo-1,4-diazaspiro[4.5]dec-3-en-1-yl]-N-(3-methylbutyl)acetamide;
3-(4-chlorophenyl)-1-{2-oxo-2-[3-(2-pyridinyl)-1-piperidinyl]ethyl}-1,4-diazaspiro[4.5]dec-3-en-2-one;
2-[3-(4-chlorophenyl)-2-oxo-1,4-diazaspiro[4.5]dec-3-en-1-yl]-N-cyclobutylacetamide;
2-[3-(4-chlorophenyl)-2-oxo-1,4-diazaspiro[4.5]dec-3-en-1-yl]-N-[(3-methyl-2-thienyl)methyl]acetamide;
3-(4-chlorophenyl)-1-{2-[2-(2-furanyl)-1-pyrrolidinyl]-2-oxoethyl}-1,4-diazaspiro[4.5]dec-3-en-2-one;
3-(4-chlorophenyl)-1-{2-oxo-2-[2-(2-thienyl)-1-pyrrolidinyl]ethyl}-1,4-diazaspiro[4.5]dec-3-en-2-one;
2-[3-(4-chlorophenyl)-2-oxo-1,4-diazaspiro[4.5]dec-3-en-1-yl]-N-(2,3-dihydro-1H-inden-2-yl)acetamide;
3-(4-chlorophenyl)-1-[2-(3-methyl-3,4-dihydro-2(1H)-isoquinolinyl)-2-oxoethyl]-1,4-diazaspiro[4.5]dec-3-en-2-one;
1-{2-oxo-2-[3-(2-pyridinyl)-1-piperidinyl]ethyl}-3-{4-[(trifluoromethyl)oxy]phenyl}-1,4-diazaspiro[4.5]dec-3-en-2-one;
2-[3-(4-chlorophenyl)-2-oxo-1,4-diazaspiro[4.5]dec-3-en-1-yl]-N-cyclohexylacetamide;
2-[3-(4-chlorophenyl)-2-oxo-1,4-diazaspiro[4.5]dec-3-en-1-yl]-N-cyclopentylacetamide;
1-{2-oxo-2-[3-(2-pyridinyl)-1-piperidinyl]ethyl}-3-[4-(trifluoromethyl)phenyl]-1,4-diazaspiro[4.5]dec-3-en-2-one;
1-{2-oxo-2-[2-(1,3-thiazol-2-yl)-1-pyrrolidinyl]ethyl}-3-[4-(trifluoromethyl)phenyl]-1,4-diazaspiro[4.5]dec-3-en-2-one;
N-butyl-2-{2-oxo-3-[4-(trifluoromethyl)phenyl]-1,4-diazaspiro[4.5]dec-3-en-1-yl}acetamide;
N-(1,1-dimethylethyl)-2-{2-oxo-3-[4-(trifluoromethyl)phenyl]-1,4-diazaspiro[4.5]dec-3-en-1-yl}acetamide;
N-(1-methylpropyl)-2-{2-oxo-3-[4-(trifluoromethyl)phenyl]-1,4-diazaspiro[4.5]dec-3-en-1-yl}acetamide;
N-butyl-2-[3-(4-chlorophenyl)-2-oxo-1,4-diazaspiro[4.5]dec-3-en-1-yl]acetamide;
2-[3-(4-chlorophenyl)-2-oxo-1,4-diazaspiro[4.5]dec-3-en-1-yl]-N-(1,1-dimethylethyl)acetamide;
2-[3-(4-chlorophenyl)-2-oxo-1,4-diazaspiro[4.5]dec-3-en-1-yl]-N-(1-methylpropyl)acetamide;
N-[2-(1-methyl-1H-pyrrol-2-yl)ethyl]-2-{2-oxo-3-[4-(trifluoromethyl)phenyl]-1,4-diazaspiro[4.5]dec-3-en-1-yl}acetamide;
2-{2-oxo-3-[4-(trifluoromethyl)phenyl]-1,4-diazaspiro[4.5]dec-3-en-1-yl}-N-[2-(2-pyridinyloxy)propyl]acetamide;
1-(2-{3-[(dimethylamino)methyl]-1-piperidinyl}-2-oxoethyl)-3-[4-(trifluoromethyl)phenyl]-1,4-diazaspiro[4.5]dec-3-en-2-one;
3-(4-chlorophenyl)-1-[2-oxo-2-(3-phenyl-1-piperidinyl)ethyl]-1,4-diazaspiro[4.5]dec-3-en-2-one;
1-{2-oxo-2-[2-(2-pyridinyl)-1-pyrrolidinyl]ethyl}-3-[4-(trifluoromethyl)phenyl]-1,4-diazaspiro[4.5]dec-3-en-2-one;
1-{2-oxo-2-[2-(3-pyridinyl)-1-piperidinyl]ethyl}-3-[4-(trifluoromethyl)phenyl]-1,4-diazaspiro[4.5]dec-3-en-2-one;
N-[(2-methylphenyl)methyl]-2-{2-oxo-3-[4-(trifluoromethyl)phenyl]-1,4-diazaspiro[4.5]dec-3-en-1-yl}acetamide;
1-[2-(1,3-dihydro-2H-isoindol-2-yl)-2-oxoethyl]-3-[4-(trifluoromethyl)phenyl]-1,4-diazaspiro[4.5]dec-3-en-2-one;
2-[3-(4-chlorophenyl)-2-oxo-1,4-diazaspiro[4.5]dec-3-en-1-yl]-N-[(2-methylphenyl)methyl]acetamide;
2-[3-(4-chlorophenyl)-2-oxo-1,4-diazaspiro[4.5]dec-3-en-1-yl]-N-(cyclohexylmethyl)acetamide; or
3-(4-chlorophenyl)-1-[2-(1,3-dihydro-2H-isoindol-2-yl)-2-oxoethyl]-1,4-diazaspiro[4.5]dec-3-en-2-one;
or a salt thereof.

34. A method of treating schizophrenia, dementia or attention deficit disorder comprising administering an effective amount of a compound of formula (I), or a pharmaceutically acceptable salt thereof, as claimed in 22.

35. A pharmaceutical composition comprising a compound of formula (I) as claimed in claim 22 or a pharmaceutically acceptable salt thereof.
